(19) United States
(12) Patent Application Publication
(10) Pub. No.: US 2025/0162878 A1
(43) Pub. Date: May 22, 2025

(54) METHOD FOR GASIFICATION OF POLYMERIC RECYCLABLE MATERIALS FOR THE LOW-EMISSION SUPPLY OF CARBON MONOXIDE USABLE FOR THE PRODUCTION OF PHOSGENE

(71) Applicant: Covestro Deutschland AG, Leverkusen (DE)

(72) Inventors: Andreas Bulan, Langenfeld (DE); Rainer Weber, Odenthal (DE); Stefanie EIDEN, Koln (DE); Gaston Zabalza, Montevideo (UY)

(21) Appl. No.: 18/841,151

(22) PCT Filed: Feb. 22, 2023
(86) PCT No.: PCT/EP2023/054477

§ 371 Date: Aug. 23, 2024

(30) Foreign Application Priority Data
Feb. 24, 2022 (EP) 22158664.7

(51) Int. Cl. C01B 32/40; C01B 32/80; C07C 263/10; C08J 11/12

(52) U.S. Cl. CPC C01B 32/40; C01B 32/80; C07C 263/10; C08J 11/12; C01B 2210/0051; C01B 2210/0053

(57) ABSTRACT

The invention relates to a method for the low-emission production of carbon monoxide (CO) for the production of phosgene, from which, for example, polycarbonates or organic isocyanates, and polyurethanes therefrom, can be produced using a process for the partial oxidation with gasification of polymeric recyclable materials. The invention further relates to a device that can be used for this purpose and to the use of said gasification process and/or polymeric recyclable materials to provide carbon monoxide for the production of phosgene.

### CROSS REFERENCE TO RELATED APPLICATIONS

This application is a U.S. national stage application, filed under 35 U.S.C. § 371, of International Application No. PCT/EP2023/054477, which was filed on Feb. 22, 2023, and which claims priority to European Patent Application No. 22158664.7, which was filed on Feb. 24, 2022. The entire contents of each are hereby incorporated by reference into this specification.

### FIELD

The invention relates to a process for low-emission production of carbon monoxide (CO) for the production of phosgene, from which for example polycarbonates or organic isocyanates and polyurethanes therefrom can be produced, using a process for partial oxidation with gasification of polymeric recyclable materials. The invention further relates to an apparatus that can be used for this purpose and to the use of said gasification process and the polymeric recyclable materials for providing carbon monoxide for the production of phosgene.

### BACKGROUND

At the end of the service life of products containing polymer materials, these are replaced with new products and normally scrapped. The total amount of plastic waste that results increases every year. About 60% of the total amount is disposed of through incineration and in landfill. When incinerated, CO₂ is emitted into the air, which contributes to global warming. Its low density means that plastic waste in landfills occupies a large volume and can from there also contribute to general pollution in rivers and seas. For this reason it is important to develop an efficient method of recycling with which the waste problem can be solved and at the same time allows fossil resources to be conserved too. The continuing growth of the plastics market means it is important to develop recycling technologies for all polymer classes in order both to reduce CO₂ emissions and to conserve fossil energies.

The processes for recycling plastic waste can be roughly divided into 3 categories:
(1) mechanical recycling: Here, the plastic waste is melted down again as is and can then be reused. This is not possible for polyurethane elastomers.
(2) chemical and thermochemical recycling: Here, the plastic waste is depolymerized into monomers or broken down into smaller molecules in order to obtain useful chemical raw materials, and
(3) thermal recycling, in which the plastic waste is converted into offgas and heat energy here.

The chemical raw materials obtained from thermochemical recycling can be used to synthesize new synthetic resins or other chemical products.

A raw material for the production of polyurethane polymers or polycarbonate polymers is carbon monoxide, which is reacted with chlorine gas to form phosgene. Thus, carbon monoxide and hydrogen are conventionally obtained from natural gas or from coal by means of reforming processes, and chlorine is obtained from electrolysis with electricity produced using fossil fuels such as oil, coal or natural gas. This gives rise to a significant amount of CO₂.

### SUMMARY

It is an object of this invention to provide a more sustainable process for producing carbon monoxide. The “sustainability” of a process is understood by those skilled in the art according to the definition of sustainability (sustainable development) coined by the UN in the Brundtland Report of the “World Commission on Environment and Development”, this being that the execution of the process in the present makes the smallest possible contribution, or none at all, to compromising the ability of future generations to meet their own needs, in particular needs in respect of the use of resources such as fossil raw materials and especially in respect of the conservation of living space, for example the protection of the earth's atmosphere.

It is thus the object of the invention to make the production of carbon monoxide more sustainable than the production methods known from the prior art, where this solution should be as easy as possible to integrate into existing production lines.

In this context it is an object of the present invention to reduce the CO₂ emissions in the provision of carbon monoxide.

Carbon monoxide can be produced from various sustainable processes. For example, through a reforming process in which natural gas from a fossil source is thus supplied with additional supply of bio-methane, carbon dioxide and/or hydrogen. The necessary energy for the ongoing reforming process is generated from the combustion of natural gas, bio-methane, hydrogen or even by electric heating. CO can also be generated from what is known as the reverse water-gas shift reaction of CO₂ and H₂, where the hydrogen originates from a water electrolysis. Another option is the electrochemical reduction of CO₂ to CO.

However, according to the process of the invention the production of CO from used polymer waste fractions and oxygen gas is effected by gasification with partial oxidation (hereinafter also referred to as gasification). The use of polymer-containing waste fractions, which can for example consist predominantly of polyurethane- or polycarbonate-containing waste fractions (hereinafter referred to as polymer waste fraction), permits the production of polycarbonate material or isocyanates and the new polyurethane material therefrom with improved sustainability. Some of the carbon is in this process recycled.

In the process of the invention, the polymer waste fraction and oxygen are brought to the partial oxidation reaction and reacted to afford a product gas mixture comprising hydrogen and CO and sometimes by-products. By-products are, in particular, hydrocarbons having 1 to 8 carbon atoms.

Also present in the product gas mixture are CO₂ and water vapor.

A further by-product obtained in the gasification process is a residual fraction that cannot be used further.

The temperature required for the gasification is achieved at least in part by partial combustion (partial oxidation) of the polymer waste fraction with an oxygen-containing gas.

As a suitable means of achieving at least one of the above objects, the invention firstly provides a process for providing carbon monoxide for the production of phosgene, preferably for the production of phosgene for the synthesis of organic isocyanate compounds, comprising at least the following steps:
a) providing an oxygen-containing gas stream containing at least 50% by weight of oxygen gas,
b) partially oxidizing material comprising at least one polymeric organic compound, said material being introduced into a reactor of an apparatus and therein being treated at least through the supply of said oxygen-containing gas stream and by heating to at least 400° C. to form a product gas, and the resulting product gas, optionally after at least one further partial oxidation step of the product gas, being discharged from the apparatus as a carbon monoxide-containing product gas stream together with particulate solid dispersed therein;
c) supplying the carbon monoxide-containing product gas stream to a purification in which at least
   c1) the carbon monoxide-containing product gas stream is, for the removal of solid, supplied to a scrubbing step in which
      (i) water is brought into contact with the carbon monoxide-containing product gas stream, as a result of which the particulate solid dispersed in the carbon monoxide-containing product gas stream forms a slag and this slag is removed and discharged,
      (ii) the carbon monoxide-containing product gas stream purified of particulate solid is discharged;
   c2) a carbon monoxide-containing product gas stream purified of particulate solid through a scrubbing step is in a drying step at least supplied to a removal of water, water is removed, and the resulting carbon monoxide-containing product gas stream is discharged,
   c3) a carbon monoxide-containing product gas stream purified through removal of water is at least supplied to a removal of carbon dioxide, carbon dioxide is removed, and the resulting carbon monoxide-containing product gas stream and carbon dioxide is discharged;
   c4) a carbon monoxide-containing product gas stream purified through removal of carbon dioxide is at least supplied to a separation unit for separating off carbon monoxide, carbon monoxide is separated off, and the resulting carbon monoxide and a hydrogen gas-containing residual gas is discharged;
   c5) optionally, a hydrogen gas-containing residual gas separated off by means of said separation unit is supplied to a residual gas treatment, hydrogen gas is separated off, and hydrogen gas and an end gas is discharged;
d) optionally synthesizing phosgene through reaction of at least chlorine gas and carbon monoxide, where the carbon monoxide that is reacted is at least the carbon monoxide from the separation unit for separating off carbon monoxide and phosgene is discharged.

### BRIEF DESCRIPTION OF DRAWINGS

FIG. 1 shows an embodiment of the process for producing carbon monoxide by gasification of a polyurethane waste fraction using oxygen from a water electrolysis for the preparation of phosgene for the synthesis of organic isocyanate.

FIG. 2 shows an embodiment of the process for producing carbon monoxide by gasification of a polycarbonate waste fraction using oxygen from a water electrolysis for the preparation of phosgene for the synthesis of polycarbonate.

### DETAILED DESCRIPTION

A solid is known to be “particulate” when it is in the form of a granular mixture of a multitude of loose, solid particles of said substance, which in turn comprises what are known as grains. A grain is a term for the particulate constituents of powders (grains are the loose, solid particles), dusts (grains are the loose, solid particles), granules (loose, solid particles are agglomerates of several grains), and other granular mixtures.

An “organic compound” contains at least one covalent carbon-hydrogen bond in the molecule. An organic isocyanate is accordingly an organic substance containing, as a chemical compound, at least one isocyanate group and at least one covalent carbon-hydrogen bond in the molecule. An organic amine is mutatis mutandis defined.

A “polymeric compound” is a molecule having a relative molecular mass (Mw) of at least 2000 g/mol, the chemical structure of which comprises mostly multiply repeating structural units derived from one or more different molecules of lower relative molecular mass. The average molar masses specified in the scope of this application for polymers or polymeric compounds are-unless explicitly otherwise stated-always weight-average molar masses Mw, which can in principle be determined by gel-permeation chromatography using an RI detector, it being expedient to perform the measurement against an external standard.

“Renewable energy” is understood by those skilled in the art to mean energy from an inexhaustible energy source, for example wind energy, hydro energy, bioenergy (e.g. conversion of biogas or biomass to power) or solar energy. Suitable renewable energy is therefore most preferably either wind power, solar energy, hydro power or mixtures thereof.

A “reactor” is a volume in which a chemical transformation, for example a partial oxidation of a polymeric organic compound of a material, takes place. For the partial oxidation this can for example be the volume of a heated vessel in which the material is contained.

The oxygen-containing gas stream provided contains at least 50% by weight of oxygen gas. Preferably, the oxygen-containing gas stream contains at least 60% by weight of oxygen gas, more preferably at least 75% by weight of oxygen gas.

The oxygen gas required for this can be withdrawn for example from a water electrolysis or an air separation plant that supplies the nitrogen, especially for the production of ammonia. Thus, the value cycle of the above polymers in respect of the carbon from the carbon monoxide can be closed. In a preferred embodiment of the process, the oxygen-containing gas stream is provided by carrying out an electrolysis of water to obtain oxygen gas and hydrogen gas and the oxygen gas from this electrolysis (22) is used to provide the oxygen-containing gas stream.

Water electrolysis can be carried out with prior art plants. Industrial systems for alkaline water electrolysis as well as for polymer electrolyte-based electrolysis, so-called PEM electrolysis, are known and commercially available. The principles of water electrolysis are described by way of example in chapter 6.3.4 in Volkmar M. Schmidt in “Elektrochemische Verfahrenstechnik” [Electrochemical process technology] (2003 Wiley-VCH-Verlag; ISBN 3-527-29958-0).

Alongside the introduction of the oxygen-containing gas stream, it may be preferable according to the invention to additionally introduce gaseous water into the at least one reactor for the partial oxidation and to carry out the partial oxidation of said material in the presence of a mixture of oxygen gas and gaseous water. In this case, it has been shown to be particularly preferable when the reactor is additionally supplied with gaseous water in a weight ratio of gaseous water to oxygen gas in the oxygen-containing gas stream of at least 0.2.

The gaseous water and the oxygen-containing gas stream can be introduced into the reactor separately from one another or the gaseous water can be mixed into the oxygen-containing gas stream before being introduced into the reactor and introduced into the reactor together with it, as the component thereof.

The material introduced into the reactor comprises according to the invention at least one polymeric organic compound. The partial oxidation should take place in the reactor as evenly and selectively as possible. An increase in these parameters can be achieved when, in a preferred embodiment, the weight ratio of the oxygen gas present in the oxygen-containing gas stream to the polymeric organic compound, in each case based on the time prior to introduction, is within a weight ratio range of from 0.4:1.0 to 1.2:1.0, preferably from 0.6:1.0 to 0.9:1.0, are introduced into the reactor.

It has been found to be advantageous when said material is introduced into the reactor in particulate form as solid particles (especially in the form of a granular mixture). A granular mixture of said material is formed from a multitude of loose, solid particles of said material, which in turn comprise what are known as grains. A grain is a term for the particulate constituents of powders (grains are the loose, solid particles), dusts (grains are the loose, solid particles), granules (loose, solid particles are agglomerates of several grains), and other granular mixtures.

The solid particles, more particularly the loose, solid particles of the granular mixture of said material introduced into the reactor, preferably have a median diameter X_{50.3} (volume average) of from 0.01 mm to 5 cm, preferably from 0.1 mm to 5 cm. The median particle size diameter X_{50.3} is determined by sieving or using a Camsizer particle size analyzer from Retsch. Preferred median particle diameters depending on the choice of reactor type are described below.

It has been found to be preferable when said material is preselected accordingly in respect of its composition before being introduced into the reactor. This results in the product gas being obtained in an improved and more consistent quality over the operating time of the partial oxidation. In this context, a minimum amount of the polymeric organic compound and also a minimum content of carbon has proved to be advantageous.

In a preferred embodiment, it is therefore advantageous when the total amount of all the polymeric organic compounds present in the material is at least 50% by weight, preferably of at least 60% by weight, more preferably of at least 75% by weight.

A further preferred embodiment of the process is characterized in that the total amount of all the polymeric organic compounds present in the material has a carbon content of at least 40.0% by weight, preferably of at least 50% by weight.

In a very particularly preferred embodiment of the process, the total amount of all the polymeric organic compounds present in the material is at least 50% by weight (preferably of at least 60% by weight, more preferably of at least 75% by weight), where the total amount of all the polymeric organic compounds present in the material has a carbon content of at least 40.0% by weight, preferably of at least 50% by weight.

A particularly preferred and therefore particularly suitable material comprises, as polymeric organic compound, those that are selected from at least one homopolymer or copolymer compound from the group cellulose, polyester, polyamide, polyurethane (PUR), polyurea, polyisocyanurate (PIR), polycarbonate, preferably from at least one homopolymer or copolymer compound from the group polyurethane (PUR) and polyisocyanurate (PIR).

In a further preferred embodiment of the process of the invention, very particularly preference as polymeric organic compounds in said material suitable for partial oxidation is given to polymeric organic compounds that have at least one repeat unit derived from monomers with a carbonyl group that in the polymeric organic compound form at least one linkage selected from urethane linkage, carbonate linkage, ester linkage, amide linkage or isocyanurate linkage. A particularly preferred embodiment of the process is a process in which the structure of the polymeric organic compound in the material supplied for the partial oxidation contains at least one repeating structural unit of the formula (I),

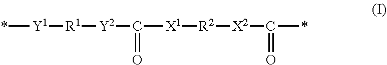

where
X¹ and X² are each independently a direct bond, O, or NH, preferably O or NH,
Y¹ and Y² are each independently O or NH,
R¹ and R² are each independently any divalent carbon-containing structural element, and a covalent bond marked with * denotes a bond to the remainder of the molecule of the polymeric organic compound.

In this most preferred embodiment it has been found to be suitable when the total amount of polymeric organic compounds having at least one repeating structural unit of the formula (I) present in the material is at least 50% by weight, preferably of at least 60% by weight, more preferably of at least 75% by weight.

When, in accordance with the formula (I), X¹ and X² are an NH group and Y¹ and Y² are O, then the polymeric organic compound contains the urethane linkage, which is very particularly preferred according to the invention. Such very particularly preferred polymeric organic compounds present in said material can be obtained through reaction at least of
i1) at least one organic isocyanate compound containing at least two to four, especially two, isocyanate groups attached to a hydrocarbon unit having 2 to 8 carbon atoms, preferably having 3 to 8 carbon atoms; with
i2) at least one organic compound having at least two hydroxy groups.

In this case, the at least one organic isocyanate compound can contain, as said hydrocarbon unit, a unit that has the number of carbon atoms stated in i1) and is derived from aliphatic hydrocarbon units, cycloaliphatic hydrocarbon units, araliphatic hydrocarbon units, aromatic hydrocarbon units or heterocyclic hydrocarbon units.

Particularly preferably, at least one compound corresponding to formula (II) is selected as said organic isocyanate compound

Q(NCO)n (II)

where n is a number from 2 to 10, preferably from 2 to 6, and Q is a radical selected from an aliphatic hydrocarbon radical having 2 to 70 carbon atoms, (preferably having 3 to 30 carbon atoms), a cycloaliphatic hydrocarbon radical having 3 to 30 carbon atoms, an aromatic hydrocarbon radical having 6 to 70 carbon atoms (preferably having 6 to 30 carbon atoms) or an araliphatic hydrocarbon radical having 6 to 70 carbon atoms (preferably having 6 to 30 carbon atoms).

It is in this case preferable when at least one diisocyanate of the formula (IIIa) or (IIIb) is selected as the at least one organic polyisocyanate compound in step i1),

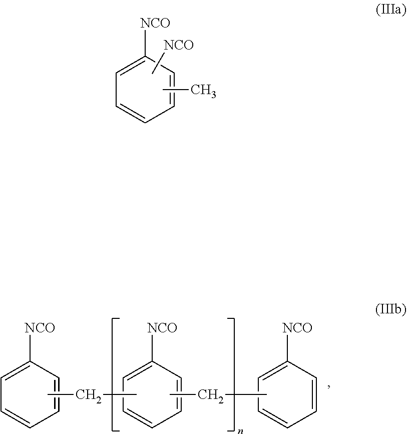

where n is a number from 0 to 8, especially from 0 to 4, more preferably from 0 to 2,

For the production of the polymeric organic compound, preference is in step i2) given to at least one organic compound having at least two hydroxy groups selected from polyester polyol, polyether polyol, polycarbonate polyol, polyetherester polyol, polyacrylate polyol, polyester polyacrylate polyol or mixtures thereof, more preferably selected from the group of polyether polyols and/or polyester polyols.

The OH value of the employed organic compound having at least two hydroxy groups or employed organic compounds having at least two hydroxy groups, in each case according to DIN 53240-1 (June 2013), is preferably from 15 to 4000 mg KOH/g. When more than just one organic compound having at least two hydroxy groups is used, the mixture of said compounds may preferably have a hydroxyl value of between 20 to 200 mg KOH/g, especially 25 to 100 mg KOH/g. In the case of a single added organic compound having at least two hydroxy groups, the OH value (or hydroxyl value) indicates the OH value of said compound. Reported OH values for mixtures relate to the number-average OH value of the mixture, calculated from the OH values of the individual components in their respective molar proportions. The OH value indicates the amount of potassium hydroxide in milligrams that is equivalent to the amount of acetic acid bound by one gram of substance on undergoing acetylation. It is determined in the context of the present descriptions according to the DIN 53240-1 standard (June 2013).

The organic compounds having at least two hydroxy groups preferably have a number-average molecular weight of ≥100 g/mol to ≤15 000 g/mol, especially ≥2000 g/mol to ≤12 000 g/mol, and more preferably ≥3500 g/mol to ≤6500 g/mol.

The number-average molar mass Mn (or molecular weight) is determined in the context of these descriptions by gel-permeation chromatography according to DIN 55672-1 of August 2007, unless explicitly stated otherwise elsewhere.

The organic compounds having at least two hydroxyl groups have a functionality of 1 to 8, where “functionality” in the context of the present invention refers to the theoretical average functionality (number of isocyanate-reactive or polyol-reactive functions in the molecule) calculated from the known input materials and proportions thereof.

Employable polyetherester polyols are compounds containing ether groups, ester groups, and OH groups. For the production of the polyetherester polyols, preference is given to using organic dicarboxylic acids having up to 12 carbon atoms, preferably aliphatic dicarboxylic acids having 4 to 6 carbon atoms or aromatic dicarboxylic acids, used individually or in a mixture. Examples include suberic acid, azelaic acid, decanedicarboxylic acid, malonic acid, phthalic acid, pimelic acid, and sebacic acid and especially glutaric acid, fumaric acid, succinic acid, adipic acid, phthalic acid, terephthalic acid, and isophthalic acid. In addition to organic dicarboxylic acids, derivatives of these acids can also be used, for example their anhydrides and also the esters and monoesters thereof with low-molecular-weight monofunctional alcohols having 1 to 4 carbon atoms. The use of proportions of the abovementioned biobased starting materials, especially of fatty acids/fatty acid derivatives (oleic acid, soybean oil, etc.), is likewise possible and can have advantages, for example in respect of storage stability of the polyol formulation, dimensional stability, fire behavior, and compressive strength of the foams.

Polyether polyols obtained by alkoxylation of starter molecules such as polyhydric alcohols are a further component used for producing polyether ester polyols. The starter molecules are at least difunctional, but may optionally also contain proportions of higher-functionality, especially trifunctional, starter molecules.

Examples of starter molecules include ethylene glycol, propylene glycol, butane-1,3-diol, butane-1,4-diol, pentene-1,5-diol, pentane-1,5-diol, hexane-1,6-diol, heptane-1,7-diol, octane-1,8-diol, decane-1,10-diol, 2-methylpropane-1,3-diol, neopentyl glycol, 2,2-dimethylpropane-1,3-diol, 3-methylpentane-1,5-diol, 2-butyl-2-ethylpropane-1,3-diol, 2-butene-1,4-diol and 2-butyne-1,4-diol, diethylene glycol, dipropylene glycol, glycerol, trimethylolpropane, and di- and trifunctional polyether polyols. The polyether polyols preferably have an OH functionality of 2 to 4 and a molecular weight Mn in the range from 62 to 4500 g/mol and especially a molecular weight Mn in the range from 62 to 3000 g/mol. Starter molecules having functionalities other than OH can also be used, alone or in a mixture.

Polyether ester polyols may also be produced by the alkoxylation, especially by ethoxylation and/or propoxylation, of reaction products obtained by the reaction of organic dicarboxylic acids and derivatives thereof as well as components having Zerewitinoff-active hydrogens, especially diols and polyols. Derivatives of such acids that may be used include, for example, their anhydrides.

The polyester polyols may for example be polycondensates of polyhydric alcohols, preferably diols, having 2 to 12 carbon atoms, preferably having 2 to 6 carbon atoms, and polycarboxylic acids, for example di, tri- or even tetracarboxylic acids or hydroxycarboxylic acids or lactones, with preference given to the use of aromatic dicarboxylic acids or mixtures of aromatic and aliphatic dicarboxylic acids. Instead of the free polycarboxylic acids it is also possible to use the corresponding polycarboxylic anhydrides or corresponding polycarboxylic esters of lower alcohols for production of the polyesters.

Useful carboxylic acids include in particular: succinic acid, glutaric acid, adipic acid, suberic acid, azelaic acid, sebacic acid, decanedioic acid, tetrahydrophthalic acid, hexahydrophthalic acid, cyclohexanedicarboxylic acid, tetrachlorophthalic acid, itaconic acid, malonic acid, 2-methylsuccinic acid, 3,3-diethylglutaric acid, 2,2-dimethylsuccinic acid, dodecanedioic acid, endomethylenetetrahydrophthalic acid, dimer fatty acid, trimer fatty acid, citric acid, trimellitic acid, benzoic acid, maleic acid, fumaric acid, phthalic acid, isophthalic acid, and terephthalic acid. It is likewise possible to use derivatives of these carboxylic acids, for example dimethyl terephthalate. The carboxylic acids may be used either individually or in a mixture. Carboxylic acids used with preference are adipic acid, sebacic acid and/or succinic acid, more preferably adipic acid and/or succinic acid.

Hydroxycarboxylic acids that may be co-used as co-reactants in the production of a polyester polyol having terminal hydroxyl groups include for example lactic acid, malic acid, hydroxycaproic acid, hydroxybutyric acid, hydroxydecanoic acid, hydroxystearic acid, and the like. Suitable lactones include caprolactone, butyrolactone, and homologs.

Also especially useful for production of the polyester polyols are biobased starting materials and/or derivatives thereof, for example castor oil, polyhydroxy fatty acids, ricinoleic acid, hydroxyl-modified oils, grapeseed oil, black cumin oil, pumpkin seed oil, borage seed oil, soybean oil, wheat germ oil, rapeseed oil, sunflower seed oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, fatty acids, hydroxyl-modified and epoxidized fatty acids and fatty acid esters, for example based on myristoleic acid, palmitoleic acid, oleic acid, vaccenic acid, petroselic acid, gadoleic acid, erucic acid, nervonic acid, linoleic acid, alpha- and gamma-linolenic acid, stearidonic acid, arachidonic acid, timnodonic acid, clupanodonic acid, and cervonic acid. Particular preference is given to esters of ricinoleic acid with polyfunctional alcohols, for example glycerol. Preference is also given to the use of mixtures of such biobased acids with other carboxylic acids, for example phthalic acids.

Examples of suitable diols are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate. Preference is given to using ethylene glycol, diethylene glycol, butane-1,4-diol, pentane-1,5-diol, hexane-1,6-diol or mixtures of at least two of said diols, especially mixtures of butane-1,4-diol, pentane-1,5-diol, and hexane-1,6-diol.

It is additionally also possible to use polyols such as trimethylolpropane, glycerol, erythritol, pentaerythritol, trimethylolbenzene or tris(hydroxyethyl) isocyanurate, with preference given to glycerol and trimethylolpropane.

The polyether polyols that may be used as an organic compound having at least two hydroxy groups are obtained by methods of preparation known to those skilled in the art, for example by anionic polymerization of one or more alkylene oxides having 2 to 4 carbon atoms with alkali metal hydroxides, such as sodium or potassium hydroxide, alkali metal alkoxides, such as sodium methoxide, sodium or potassium ethoxide or potassium isopropoxide, or aminic alkoxylation catalysts, such as dimethylethanolamine (DMEA), imidazole and/or imidazole derivatives, or DMC catalysts using at least one starter molecule having 2 to 8, preferably 2 to 6, attached reactive hydrogen atoms.

Examples of suitable alkylene oxides are tetrahydrofuran, 1,3-propylene oxide, 1,2- and 2,3-butylene oxide, styrene oxide and preferably ethylene oxide and 1,2-propylene oxide. The alkylene oxides may be used individually, alternately one after the other, or as mixtures. Preferred alkylene oxides are propylene oxide and ethylene oxide, with particular preference given to copolymers of propylene oxide with ethylene oxide. The alkylene oxides may be reacted in combination with CO₂.

Examples of useful starter molecules include: water, organic dicarboxylic acids, such as succinic acid, adipic acid, phthalic acid, and terephthalic acid, aliphatic and aromatic, optionally N-mono, N,N- and N,N′-dialkyl-substituted diamines having 1 to 4 carbon atoms in the alkyl radical, such as optionally mono- and dialkyl-substituted ethylenediamine, diethylenetriamine, triethylenetetramine, 1,3-propylenediamine, 1,3- and 1,4-butylenediamine, 1,2, 1,3, 1,4, 1,5, and 1,6-hexamethylenediamine, phenylenediamines, 2,3, 2,4, and 2,6-tolylenediamine, and 2,2′,2,4′, and 4,4′-diaminodiphenylmethane.

Preference is given to using dihydric or polyhydric alcohols, such as ethanediol, propane-1,2- and -1,3-diol, diethylene glycol, dipropylene glycol, butane-1,4-diol, hexane-1,6-diol, paraformaldehyde, triethanolamine, bisphenols, glycerol, trimethylolpropane, pentaerythritol, sorbitol, and sucrose.

Polycarbonate polyols that may be used are polycarbonates having hydroxyl groups, for example polycarbonate diols. These are formed in the reaction of carbonic acid derivatives, such as diphenyl carbonate, dimethyl carbonate or phosgene, with polyols, preferably diols.

Examples of such diols are ethylene glycol, propane-1,2- and -1,3-diol, butane-1,3- and -1,4-diol, hexane-1,6-diol, octane-1,8-diol, neopentyl glycol, 1,4-bishydroxymethylcyclohexane, 2-methylpropane-1,3-diol, 2,2,4-trimethylpentane-1,3-diol, dipropylene glycol, polypropylene glycols, dibutylene glycol, polybutylene glycols, bisphenols, and lactone-modified diols of the abovementioned type.

Also employable instead of or in addition to pure polycarbonate diols are polyether-polycarbonate diols obtainable for example by copolymerization of alkylene oxides, for example propylene oxide, with CO₂.

As compounds having isocyanate-reactive hydrogen atoms, it is also possible to use polymer polyols, PHD polyols, and PIPA polyols as an organic compound having at least two hydroxy groups. Polymer polyols are polyols containing proportions of solid polymers produced by free-radical polymerization of suitable monomers such as styrene or acrylonitrile in a base polyol. PHD (polyhydrazodicarbonamide) polyols are produced for example by in-situ polymerization of an isocyanate or an isocyanate mixture with a diamine and/or hydrazine (or hydrazine hydrate) in a polyol, preferably a polyether polyol. The PHD dispersion is preferably produced by reaction of an isocyanate mixture composed of 75% to 85% by weight of tolylene 2,4-diisocyanate (2,4-TDI) and 15% to 25% by weight of tolylene 2,6-diisocyanate (2,6-TDI) with a diamine and/or hydrazine hydrate in a polyether polyol produced by alkoxylation of a trifunctional starter (for example glycerol and/or trimethylolpropane). PIPA polyols are polyether polyols modified with alkanolamines by polyisocyanate polyaddition, where the polyether polyol preferably has a functionality of from 2.5 to 4.0 and a hydroxyl value of from 3 mg KOH/g to 112 mg KOH/g (molecular weight from 500 g/mol to 18 000 g/mol).

It is also possible to use isocyanate-reactive substances having a cell opening effect, for example copolymers of ethylene oxide and propylene oxide having an excess of ethylene oxide or aromatic diamines such as diethyltoluenediamine.

For the production of polyurethane foams in the cold-cure foam process, polyethers having at least two hydroxyl groups and an OH value of 20 to 50 mg KOH/g are in a further embodiment employed as an organic compound having at least two hydroxy groups, wherein at least 80 mol % of the OH groups are primary OH groups (determination by 1H NMR (for example Bruker DPX 400, deuterochloroform)). It is particularly preferable when the OH value is 25 to 40 mg KOH/g, very particularly preferably 25 to 35 mg KOH/g.

In addition to the structural unit of the formula (I) defined above, the polymeric organic compound of the material may contain isocyanurate structural units of the formula

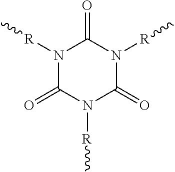

where R is a divalent hydrocarbon radical, especially a divalent aromatic hydrocarbon radical. Corresponding isocyanurate structural units are found, for example, to a major extent in rigid polyurethane foams.

The material comprising said polymeric organic compound is preferably a foam, more preferably a polyurethane foam. When the material is present in the form of a polyurethane foam, it is in turn preferably a flexible polyurethane foam or a rigid polyurethane foam.

When, in accordance with the formula (I), X¹ and X² are an O group and Y¹ and Y² are O, then the polymeric organic compound contains the carbonate linkage, which is very particularly preferred according to the invention.

Polymeric organic compounds of this embodiment that are suitable for the process of the invention are for example aromatic polycarbonates and/or aromatic polyestercarbonates. These are known from the literature or can be produced by processes known from the literature (for the production of aromatic polycarbonates see for example Schnell, “Chemistry and Physics of Polycarbonates”, Interscience Publishers, 1964, and also DE-AS 1 495 626, DE-A 2 232 877, DE-A 2 703 376, DE-A 2 714 544, DE-A 3 000 610, DE-A 3 832 396; for production of aromatic polyestercarbonates see for example DE-A 3 007 934).

The aromatic polycarbonates usable as a corresponding polymeric organic compound are produced for example through reaction of compounds having at least two hydroxyl groups, especially diphenols, with carbonyl halides, preferably phosgene and/or with aromatic dicarboxylic acid dihalides, preferably dihalides of benzenedicarboxylic acid, by the interfacial process, optionally using chain terminators, for example monophenols, and optionally using trifunctional or more than trifunctional branching agents, for example triphenols or tetraphenols. Also possible is production via a melt polymerization process by reaction of compounds having at least two hydroxyl groups, especially diphenols, with for example diphenyl carbonate.

According to the invention, it is possible to advantageously use said materials of this kind in which the polymeric organic compound is at least one compound obtained through reaction of at least the compounds (i) at least one aromatic compound having at least two hydroxyl groups, particularly preferably bisphenol A, and (ii) phosgene or diphenyl carbonate.

For the production of the polycarbonate-containing compound it is preferable to use at least one aromatic compound having at least two hydroxyl groups that is selected from the general formula (IV),

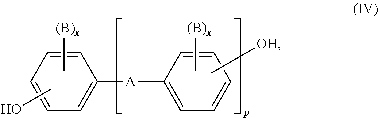

where
A is a single bond, C1 to C₅ alkylene, C₂ to C₅ alkylidene, C₅ to C₆ cycloalkylidene, —O—, —SO, —CO—, —S—, —SO₂, C₆ to Cl₂ arylene, onto which further aromatic rings optionally containing heteroatoms may be fused,
   or a radical of the formula (V) or (VI)

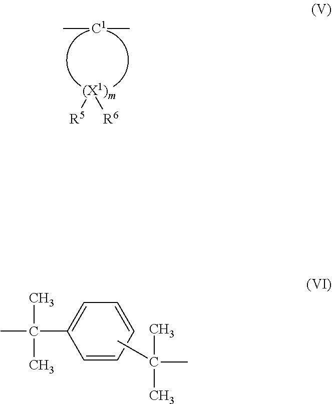

B is in each case C1 to Cl₂ alkyl, preferably methyl, halogen, preferably chlorine and/or bromine,
x is in each case independently 0, 1 or 2,
p is 1 or 0, and
R⁵ and R⁶ can be selected individually for each X¹ and are each independently hydrogen or C₁ to C₆ alkyl, preferably hydrogen, methyl or ethyl,
X¹ is carbon and
m is an integer from 4 to 7, preferably 4 or 5, with the proviso that, on at least one atom X¹, R⁵ and R⁶ are both alkyl.

Preferred aromatic compounds having at least two hydroxyl groups are hydroquinone, resorcinol, dihydroxydiphenols, bis(hydroxyphenyl)-C₁ to C₅ alkanes, bis(hydroxyphenyl)-C₅ to -C₆ cycloalkanes, bis(hydroxyphenyl)ethers, bis(hydroxyphenyl)sulfoxides, bis(hydroxyphenyl) ketones, bis(hydroxyphenyl)sulfones and α,α-bis(hydroxyphenyl)diisopropylbenzenes, and ring-brominated and/or ring-chlorinated derivatives thereof.

Particularly preferred aromatic compounds having at least two hydroxyl groups are 4,4′-dihydroxydiphenyl, bisphenol A, 2,4-bis(4-hydroxyphenyl)-2-methylbutane, 1,1-bis(4-hydroxyphenyl)cyclohexane, 1,1-bis(4-hydroxyphenyl)-3,3,5-trimethylcyclohexane, 4,4′-dihydroxydiphenyl sulfide, 4,4′-dihydroxydiphenyl sulfone, and also the di- and tetrabrominated or chlorinated derivatives thereof, for example 2,2-bis(3-chloro-4-hydroxyphenyl) propane, 2,2-bis(3,5-dichloro-4-hydroxyphenyl) propane or 2,2-bis(3,5-dibromo-4-hydroxyphenyl) propane. Particular preference is given to 2,2-bis(4-hydroxyphenyl) propane (bisphenol A).

The aromatic compounds having at least two hydroxyl groups may be used individually or as any desired mixtures. The aromatic compounds having at least two hydroxyl groups are known from the literature or can be obtained by methods known from the literature.

In a further embodiment of the invention, employable thermoplastic aromatic polycarbonates have average molecular weights (weight-average Mw, measured by GPC (gel-permeation chromatography) using a polycarbonate standard based on bisphenol A) of preferably 20 000 to 40 000 g/mol, more preferably 24 000 to 32 000 g/mol, particularly preferably 26 000 to 30 000 g/mol.

The thermoplastic aromatic polycarbonates may be branched in a known manner and preferably through incorporation of 0.05 to 2.0 mol %, based on the sum total of aromatic compound having at least two hydroxyl groups used, of trifunctional or more than trifunctional compounds, for example ones having three or more phenolic groups.

Preference is given to using linear polycarbonates, more preferably ones based on bisphenol A.

Both homopolycarbonates and copolycarbonates are suitable as polymeric organic compounds. For the production of copolycarbonates employable with preference according to the invention it is also possible to use 1% to 25% by weight, preferably 2.5% to 25% by weight, based on the total amount of aromatic compound having at least two hydroxyl groups to be used, of polydiorganosiloxanes having hydroxyaryloxy end groups. These are known (U.S. Pat. No. 3,419,634) and may be produced by processes known from the literature. Likewise suitable are polydiorganosiloxane-containing copolycarbonates; the production of polydiorganosiloxane-containing copolycarbonates is described for example in DE-A 3 334 782.

Aromatic dicarboxylic acid dihalides for the production of aromatic polyestercarbonates are preferably the diacid dichlorides of isophthalic acid, terephthalic acid, diphenyl ether 4,4′-dicarboxylic acid, and naphthalene-2,6-dicarboxylic acid.

The aromatic polyestercarbonates may be linear or they may be branched in a known manner (see DE-A 2 940 024 and DE-A 3 007 934), preference being given to linear polyestercarbonates.

The partial oxidation of the material used is carried out in the reactor at a temperature of at least 400° C. Preferably, the partial oxidation of the material in the reactor is carried out at a temperature in a temperature range of from 600° C. to 1500° C., especially from 850° C. to 1400° C., more preferably from 1100° C. to 1300° C.

The partial oxidation of the material used is in a preferred embodiment carried out at an absolute pressure of more than 1 bar, preferably at an absolute pressure within a range from 2 to 80 bar, more preferably at an absolute pressure within a range from 2 to 50 bar.

The partial oxidation of the material can be carried out in at least one of the following three reactors: reactor for entrained-flow gasification, reactor for fluidized-bed gasification, and reactor for fixed-bed gasification.

For use in a reactor for entrained-flow gasification, the polymeric organic compounds, or the material in which they are present, must be ground to a grain size having a median particle diameter X_{50.3} of <0.1 mm (dust). The supply to the reactor is effected either pneumatically or as a slurry. The greatest limitation of this type of partial oxidation for the chemical recycling of waste is the grindability and conveyability of starting materials from heterogeneous waste. A thermal treatment of biomass (torrefaction) at 200-300° C. with exclusion of O₂ is used to produce a “biochar” with similar grindability to coal. In another variant, an upstream pyrolysis can be used to produce a pumpable pyrolysis oil. The oil obtained by waste pyrolysis can be partially oxidized either directly or in the form of a slurry mixed with the solid pyrolysis residue (pyrolysis coke). This process configuration was developed by Noel (Noell-Konversionsverfahren zur Verwertung und Entsorgung von Abfällen [Noell conversion process for the recovery and disposal of waste], Jürgen Carl EF-Verl. für Energie-und Umwelttechnik, 1994. ISBN: 3924511829).

Another variant is the use of a reactor for fluidized-bed gasification. The gasification of waste in fluidized-bed reactors is widely known, with the technologies behind EBARA (Showa Denko, Japan) and ENERKEM (Enerkem, Edmonton, Canada) and the large-scale demonstration of the high-temperature Winkler gas production (HTW) by Rheinbraun AG (now RWE) from 1993 to 1997 in Berrenrath, Germany. The pretreatment for feeding the polymeric organic compounds or the material in which they are present into the reactor requires comminution to a median particle diameter X_{50.3} of 30-80 mm. The feed into the reactor tank is via screw conveyors, which limits the gasifier pressure to a maximum of 10 bar. The ENERKEM and EBARA technologies permit the gasification of high-caloric waste (plastic waste or plastic-rich substitute fuels). Fluidized-bed gasifiers are operated at mild temperatures of 700-950° C., well below the ash melting point of the feedstock, in order to avoid caking and agglomeration in the reactor. A further advantage of this mild reactor temperature is the incomplete carbon turnover in the fluidized bed. Furthermore, the crude gas from reactors for fluidized-bed gasifiers typically contains significant amounts of methane and other hydrocarbons. To compensate for this and to ensure a high syngas yield of H₂ and CO, the ENERKEM and EBARA processes employ a second high-temperature stage for partial oxidation (approx. 1400° C.) arranged directly downstream of the fluidized bed, in order to melt fly ash and convert hydrocarbons in the product gas from the first stage into the final carbon monoxide-containing product gas stream. ENERKEM calls this second stage “thermoreformer”, while EBARA refers to a “high-temperature gasification furnace”. The high temperature in this second partial oxidation stage increases CO₂ production.

In a further embodiment of the process of the invention, the partial oxidation of the material is preferably carried out in at least two steps, wherein in a first step a partial oxidation of the material is carried out in a first reactor at a temperature in a temperature range of 400° C. to 800° C. and the product gas from the first reactor is transferred to a second reactor where, in a further step with supply of an oxygen-containing gas stream, it is subjected to a partial oxidation at a temperature of more than 800° C., preferably at a temperature within a range of from 1300° C. to 1500° C., to obtain the carbon monoxide-containing product gas stream.

In fixed-bed gasification a distinction is made between processes with dry ash and discharge with liquid slag. The British Gas Lurgi (BGL) process (British Gas/Lurgi slagging gasifier, published on: 1993 Aug. 30) has been successfully employed for co-gasification of up to 80% waste (substitute fuels, modernized municipal solid waste and industrial waste, shredder light fraction, mixed plastic waste, waste, sewage sludge) with lignite and coal.

The pretreatment of waste destined for partial oxidation in fixed-bed gasifiers with slags necessitates granulation, compression or pressing. The waste feedstock is then introduced into the reactor via a lock system at the head of the reactor (gasifier). This allows the reactor to be operated at pressures of up to 40 bar, reducing the compressor for subsequent gas treatment and chemical synthesis. Near the base of the reactor, the pipes heat the feedstock to well above the ash melting point, forming a slag bath. Vitrified, almost carbon-free slag (<1% by weight) is discharged discontinuously into a water-filled chamber. The product gas moves upward through the reactor (gasifier) and, as a second step of the partial oxidation, passes through a post-gasification zone in which additional oxygen and preferably additionally gaseous water (steam) is introduced in order to increase the temperature to 1000-1150° C. and convert methane and hydrocarbons, prior to it exiting the reactor as a carbon monoxide-containing product gas stream.

The carbon monoxide-containing product gas from the partial oxidation that is obtainable by the process of the invention comprises, in addition to carbon monoxide, mostly proportions of CO₂, as well as water, hydrogen gas, and methane. Furthermore, particulate solids are additionally dispersed in the carbon monoxide-containing product gas from the partial oxidation that is obtainable by the process of the invention.

The carbon monoxide-containing product gas stream is accordingly subsequently supplied to a purification in which, in order of priority, to separate solid material, the carbon monoxide-containing product gas stream is first supplied to a scrubbing step, in which
(i) water is brought into contact with the carbon monoxide-containing product gas stream, as a result of which the particulate solid dispersed in the carbon monoxide-containing product gas stream forms a slag and this slag is removed and discharged,
(ii) the carbon monoxide-containing product gas stream purified of particulate solid is discharged.

In the scrubbing step, the carbon monoxide-containing product gas withdrawn from the partial oxidation can initially be introduced into the scrubbing step at a temperature of more than 400° C. In this case, said product gas is, after having been contacted with water, cooled and the water that evaporates in the process, for example as condensate, is separated and optionally returned to the process.

The carbon monoxide-containing product gas stream that has been discharged from the scrubbing step and purified of particulate solid preferably has a temperature of not more than 100° C., more preferably of not more than 80° C.

It has proved to be particularly preferable when, for the contacting, the water is sprayed into the carbon monoxide-containing product gas stream during the scrubbing step.

Carbon monoxide-containing product gas purified of particulate solid is-optionally after performing further optional purification steps different from steps c1) to c5)—according to order of priority c2) next supplied to at least a removal of water in a drying step, water is removed, and the resulting carbon monoxide-containing product gas stream is discharged.

In a water removal unit, the water is separated, for example, by cooling the carbon monoxide-containing product gas withdrawn from the partial oxidation and separating the water, for example as condensate.

The separated water can be supplied to scrubbing step c1).

A carbon monoxide-containing product gas stream purified through removal of water is-optionally after performing further optional purification steps different from steps c1) to c5)—according to order of priority c3) next supplied to at least a removal of carbon dioxide, carbon dioxide is removed, and the resulting carbon monoxide-containing product gas stream and carbon dioxide are discharged. In this case, the carbon dioxide discharged can be released to the environment or, as described later, introduced into the partial oxidation in addition to the oxygen-containing gas and recycled there.

The removal of CO₂ can be executed as an “amine scrubbing”, where the carbon monoxide-containing process gas here undergoes especially the generally known scrubbing of the gas mixture by the principle of chemisorption with amines, such as monoethanolamine (MEA), diethanolamine (DEA), methyldiethanolamine (MDEA) or diglycolamine (DGA), which achieves a high purity of the purified gas mixture in an absorption column.

To reduce the CO₂ footprint, it is in a further embodiment of the present invention very particularly preferable when the removed carbon dioxide discharged in step c3) is recycled to a reactor for the partial oxidation and introduced into this reactor.

In one embodiment of the abovementioned CO₂ recycling, the carbon dioxide is preferably added to the partial oxidation in an amount of not more than 1 kg per kg of polymeric organic compound, preferably of not more than 0.9 kg per kg of polymeric organic compound, more preferably of 0.8 kg per kg of polymeric organic compound.

If the partial oxidation of the material is carried out in at least two reactors, the removed carbon dioxide discharged in step c3) is recycled at least to the first reactor for the partial oxidation and introduced into this reactor.

Although particular preference is given to CO₂ recycling, CO₂ for the partial oxidation can also be supplied from a different CO₂ source, for example the CO₂ emitted in the provision of thermal energy from the combustion chamber of a heating element, or CO₂ from an external CO₂ source. This can also be done in addition to the CO₂ recycling mentioned above.

A further CO₂ source preferably used is at least one external CO₂ source that contributes CO₂ which is not emitted by the process of the invention. An external CO₂ source would be for example the CO₂ that in cement production, is obtained in H₂ production for ammonia synthesis, forms in the offgas on combustion of fuels (e.g. waste combustion), or CO₂ extracted from the air. This CO₂ from an external CO₂ source will in a preferred embodiment of the process of the invention be effected by absorption of a CO₂ content from (i) process gases or offgases that is selected from at least one process selected from cement production, H₂ production, incineration, and/or (ii) from air by introduction into alkali metal hydroxide solution, for example potassium hydroxide solution. This results in the formation of potassium hydrogen carbonate, which can then be thermally decomposed back to CO₂ and potassium hydroxide. The CO₂ released here is then supplied to the partial oxidation for the synthesis of carbon monoxide. To provide the thermal energy needed for the release of CO₂, it is possible, for example, to use the thermal energy generated from the partial oxidation.

The CO₂ can optionally undergo further purification steps in order to produce CO₂ of sufficient purity for said partial oxidation.

A carbon monoxide-containing product gas stream purified through removal of carbon dioxide is—optionally after performing further optional purification steps different from steps c1) to c5)—according to the order of priority next supplied to at least a separation unit for separating off carbon monoxide c4), carbon monoxide is separated off, and the resulting carbon monoxide and a hydrogen gas-containing residual gas are discharged.

This forms preferably at least one gas stream, wherein the gas at 25° C. and 1013 mbar contains at least 95% by volume of carbon monoxide, more preferably at least 99% by volume of carbon monoxide, which is discharged as carbon monoxide. Said carbon monoxide-containing product gas stream purified of carbon dioxide, is, in the separation unit for separating off carbon monoxide (hereinafter referred to also as the H₂—CO separation unit), preferably first separated into two gas streams. This gives rise to a gas in the form of a gas stream containing at least 95% by volume (preferably at least 99% by weight) of carbon monoxide, a further gas in the form of a gas stream having hydrogen as its major constituent and carbon monoxide and methane inter alia. The further gas is also referred to as residual gas from the H₂—CO separation or, if there is no residual gas treatment, as end gas. A H₂—CO separation unit that works by this principle of separation is called the coldbox.

The hydrogen-containing residual gas from the H₂—CO separation can be subjected to a subsequent residual gas treatment to concentrate the hydrogen. In this case, a hydrogen gas-containing residual gas separated off by means of said separation unit for separating off carbon monoxide is supplied to a residual gas treatment, hydrogen gas is separated off, and hydrogen gas and an end gas is discharged. Processing by the residual gas treatment affords a gas enriched with hydrogen gas in the form of a gas stream and another gas-what is referred to as the residual gas from the residual gas treatment or end gas—in the form of a gas stream, the latter comprising a mixture of CO, methane and a smaller amount of hydrogen than the hydrogen gas-enriched gas.

The hydrogen gas of the process of the invention separated off through the separating-off of carbon monoxide can be used for the hydrogenation of nitro compounds in the production of amines as a raw material for isocyanate synthesis. In a preferred embodiment of the process, the gaseous hydrogen separated from the residual gas of the H₂—CO separation is, after the separation of hydrogen in the H₂—CO separation, especially using a coldbox, after further residual gas treatment fed into a hydrogenation of organic nitro compound for the production of organic amine as a raw material for organic isocyanate as hydrogen prominent therefrom.

As mentioned, in a preferred embodiment of the process there is additionally a step d) of synthesizing phosgene through reaction of at least chlorine gas and carbon monoxide, where the carbon monoxide that is reacted is at least the carbon monoxide from purification step c) and phosgene is discharged. In particular, said purification is in this case preferably carried out according to an embodiment suitable for a high degree of purity and the remaining carbon monoxide from the purification is fed into the phosgene synthesis.

For the provision of the chlorine for the phosgene synthesis carried out in this embodiment, those skilled in the art will be well acquainted with the production of chlorine gas from electrochemical oxidation by the electrolysis of hydrochloric acid with a gas-diffusion electrode (also referred to as the HCl ODC electrolysis process (ODC=oxygen-depleting electrode); for a suitable electrolysis cell see U.S. Pat. No. 6,022,634 A, WO 03/31690 A1), the production of chlorine gas from hydrochloric acid diaphragm electrolysis (see EP 1 103 636 A1), the production of chlorine gas from thermocatalytic gas-phase oxidation (see WO 2012/025483 A2), and the production of chlorine from chloralkali electrolysis (see WO 2009/007366 A2). Reference is made expressly and in full to the content of the abovementioned documents cited in connection with the production of chlorine gas. The chlorine required for the synthesis of phosgene is in a preferred variant of this embodiment of the process produced electrolytically, especially through electrochemical oxidation by hydrochloric acid electrolysis with a gas-diffusion electrode, through electrochemical oxidation by hydrochloric acid diaphragm electrolysis or through electrochemical oxidation by chloralkali electrolysis. It is in this case in turn particularly preferable when said electrochemical oxidation is in each case carried out using electricity generated from renewable energy, especially from renewable energy in the form of wind power, solar energy or hydro power.

The phosgene formed by the phosgene synthesis is in a particularly preferred embodiment of the process used in a further step e) for the production of organic isocyanate (9), wherein the phosgene (8a) from the phosgene synthesis (8) is reacted with at least one organic amino compound (20a) and at least organic isocyanate (9a) is discharged. Preferably, at least organic isocyanate and hydrogen chloride are discharged.

In general, preference is in this embodiment given to those processes of the invention in which the organic isocyanate obtained contains at least two isocyanate groups. For this purpose, the reactant used in the synthesis is in turn preferably organic amine having at least two amino groups. More preferably, the organic isocyanate obtained contains at least two isocyanate groups and has a molar mass of not more than 1000 g/mol, especially of not more than 800 g/mol.

Organic amines used with very particular preference are selected from tolylenediamine (TDA), methylenedi (phenylamine) (MDA) (preferably selected in turn from diphenylmethane-2,2′-diamine, diphenylmethane-2,4′-diamine, diphenylmethane-4,4′-diamine or mixtures thereof), hexamethylenediamine, isophoronediamine, 1,3-bis(aminomethyl)benzene, cyclohexyldiamine or mixtures thereof, where TDA, MDA or mixtures thereof are very particularly preferred organic isocyanates.

Organic isocyanates obtained with very particular preference are selected from tolylene diisocyanate (TDI), methylene di(phenyl isocyanate) (MDI) (preferably selected in turn from diphenylmethane 2,2′-diisocyanate, diphenylmethane 2,4′-diisocyanate, diphenylmethane 4,4′-diisocyanate or mixtures thereof), hexamethylene diisocyanate (HDI), isophorone diisocyanate (IPDI), 1,3-bis(isocyanatomethyl)benzene (XDI), cyclohexyl diisocyanate (CHDI) or mixtures thereof, where TDI, MDI or mixtures thereof are very particularly preferred organic isocyanates.

Furthermore, in the embodiment with synthesis of organic isocyanate it is preferable that hydrogen chloride is additionally formed alongside said organic isocyanate. It is in turn preferable that this hydrogen chloride is supplied as a reactant for the production of chlorine for said phosgene synthesis. The necessary separation-off and purification of the hydrogen chloride formed in the production of isocyanate can then be accomplished through oxidative reaction in a thermocatalytic gas-phase oxidation with oxygen to chlorine and water, and optionally using O₂ from water electrolysis.

A further alternative approach to utilizing the hydrogen chloride as a reactant for the production of chlorine is the reaction of the hydrogen chloride with water to hydrochloric acid and subsequent electrochemical oxidation of the hydrochloric acid to chlorine and optionally hydrogen. This aqueous hydrochloric acid is in particular supplied to the previously described electrochemical oxidation by hydrochloric acid electrolysis with a gas-diffusion electrode, electrochemical oxidation by hydrochloric acid diaphragm electrolysis or electrochemical oxidation by chloralkali electrolysis). O₂ required in the case of hydrochloric acid electrolysis with a gas-diffusion electrode can be obtained from water electrolysis.

The hydrogen chloride resulting from isocyanate production can also, after purification and absorption in water, be converted into hydrochloric acid and the resulting hydrochloric acid sold on the market for various uses.

A particularly preferred embodiment of the process additionally involves the performance of a synthesis of phosgene through reaction of at least chlorine gas and the carbon monoxide obtained previously by purification of said carbon monoxide-containing product gas from step c);
a production of isocyanate through reaction of the phosgene obtained with at least one organic amine to form at least one organic isocyanate compound and hydrogen chloride;
a separation and purification of the hydrogen chloride formed in the production of isocyanate and a subsequent oxidative reaction of the hydrogen chloride, selected from
   a reaction of hydrogen chloride in a thermocatalytic gas-phase oxidation with oxygen to chlorine and water, optionally using oxygen from water electrolysis,
   a reaction of hydrogen chloride with water to form hydrochloric acid and subsequent electrochemical oxidation of hydrochloric acid to chlorine and optionally hydrogen;
following the oxidative conversion of the hydrogen chloride, a supply of the chlorine that is formed to said synthesis of phosgene, optionally with the supply to said synthesis of phosgene of an additional amount of chlorine that is derived from a chloralkali electrolysis.

For all embodiments of the process that include a synthesis of phosgene as a step, it is further preferable to carry out an electrochemical oxidation (electrolysis) for chlorine production using electricity generated from renewable energy, especially electricity optionally obtained through the use of wind power, solar energy or hydro power.

The organic amines used for isocyanate production in the embodiment of the process are in turn preferably provided by an additional step of hydrogenation of organic nitro compounds with gaseous hydrogen. Hydrogen used for this preferably comes from water electrolysis and/or from the purification of the carbon monoxide-containing product gas in the form of separated hydrogen after the steps of separating off carbon monoxide c4) and residual gas treatment c5).

In a particularly preferred embodiment of the process of the invention, hydrogen is fed from a water electrolysis to the hydrogenation of organic nitro compound for the production of organic amine. Particularly preferably, the hydrogen is in this case formed by water electrolysis using electricity from renewable energy (especially from renewable energy in the form of wind power, solar energy or hydro power) and supplied to a step for the hydrogenation of at least one organic nitro compound, where the at least one organic amine obtained in the hydrogenation of said nitro compound is reacted with phosgene to obtain at least one organic isocyanate.

It is particularly preferable according to the invention to supply at least the hydrogen separated from the carbon monoxide-containing product gas during purification to a step of hydrogenation of at least one organic nitro compound. This likewise affords at least one organic amine for the production of organic isocyanate.

The organic isocyanates obtained can in an additional step of the process be reacted with at least one organic compound having at least two hydroxy groups, especially at least one polyester polyol or polyether polyol, to form polyurethane materials. For production, the preferred embodiments for the provision of polyurethane material are here those described above for characterizing the preferably suitable polymeric organic compound in the material used for the partial oxidation.

Correspondingly produced polyurethane materials are for example in the form of foams, coatings, insulating compound and components of commercial products. At the end of their life cycle, they can serve as material for the partial oxidation in the process of the invention.

The present invention further provides an apparatus with which the process of the invention can in particular be carried out. This is an apparatus for providing carbon monoxide for the production of phosgene, preferably for the production of phosgene for synthesizing organic isocyanate compound, comprising at least one metering apparatus for feeding in material, at least one apparatus for the partial oxidation, and at least one purification apparatus for carbon monoxide-containing product gas, characterized in that
i) said apparatus for the partial oxidation comprises
at least one outlet for a carbon monoxide-containing product gas stream
and
at least one reactor for the partial oxidation of the material having at least one control capable of setting the reactor to a temperature of at least 400° C., where the reactor comprises at least one inlet for an oxygen-containing gas stream and at least one inlet for material and at least one outlet for product gas that is different therefrom, where the inlet for an oxygen-containing gas stream is in fluid connection with a source of an oxygen-containing gas stream containing at least 50% by weight of oxygen gas, and the outlet for product gas of the reactor is in fluid connection, optionally at least via at least one interposed reactor for the partial oxidation of the product gas, with the outlet for the carbon monoxide-containing product gas stream; and,
ii) the metering apparatus and said apparatus for the partial oxidation are arranged with respect to one another and configured such that the metering apparatus is in fluid connection, via at least one supply line, with the inlet for material of the reactor for the partial oxidation of the material; and
iii) a purification apparatus for carbon monoxide-containing product gas that comprises at least one outlet for carbon monoxide and at least one inlet for the carbon monoxide-containing product gas stream, said inlet being in fluid connection with the outlet for the carbon monoxide-containing product gas stream of the apparatus for the partial oxidation, the purification apparatus being configured such that the carbon monoxide-containing product gas stream can be passed for purification through modules listed below in the specified order of priority;
iii-1) at least one gas-scrubbing apparatus, comprising at least one inlet for the carbon monoxide-containing product gas stream, at least one inlet for water, at least one scrubbing tank in which water and carbon monoxide-containing product gas stream are brought together, and at least one outlet for carbon monoxide-containing product gas stream and at least one outlet for slag, where the scrubbing tank and the outlet for slag are in fluid connection and the scrubbing tank and the outlet for carbon monoxide-containing product gas stream are in fluid connection;
iii-2) at least one drying apparatus, comprising at least one inlet for carbon monoxide-containing product gas stream, at least one cooling apparatus suitable for cooling the carbon monoxide-containing product gas stream, at least one outlet for carbon monoxide-containing product gas stream, and at least one outlet for liquid condensate,
iii-3) at least one apparatus for removal of carbon dioxide, comprising at least one inlet for carbon monoxide-containing product gas stream, at least one outlet for carbon monoxide-containing product gas stream and, in addition, at least one outlet for carbon dioxide,
iii-4) at least one separating unit for separating off carbon monoxide, comprising at least one inlet for carbon monoxide-containing product gas stream, at least one outlet for carbon monoxide and, in addition, at least one outlet for hydrogen gas-containing residual gas.

A “fluid connection” is in accordance with the invention understood as meaning a part of the device that connects parts of the system to one another and through which a substance that may be in any state of matter can be transported from one plant component to another plant component by a material stream, for example a feed conduit in the form of a pipe, which can be interrupted by further plant components, or a feed conduit in the form of a conveyor, for example a chute, conveyor screw or conveyor belt for solids.

Preferably, the reactors of the apparatus for the partial oxidation are selected from at least one reactor of the group formed from reactors for entrained-flow gasification, reactors for fluidized-bed gasification, and reactors for fixed-bed gasification.

The source of an oxygen-containing gas stream containing at least 50% by weight of oxygen gas is preferably an apparatus for water electrolysis, the outlet for oxygen gas of which is in fluid connection with the inlet for the oxygen-containing gas stream present in the reactor. By analogy with the process described above, it is in a preferred embodiment of the apparatus likewise preferable when the apparatus of the invention additionally comprises an apparatus for water electrolysis that, in addition to at least one anode and at least one cathode, additionally has at least one inlet for water, at least one outlet for hydrogen gas, at least one outlet for oxygen gas, and is connected to a source for electricity from renewable energy, the outlet for hydrogen gas of which being in fluid connection with a hydrogen gas inlet of an apparatus for the hydrogenation of organic nitro compound.

The preferred embodiments in each case of the gas-scrubbing apparatus, the drying apparatus, the apparatus for removal of carbon dioxide, and the separation unit for separating off carbon monoxide are designed as described under the subject matter of the process of the invention (vide supra).

It has been found to be preferable when, in a preferred embodiment, the apparatus is characterized in that the purification apparatus additionally comprises at least one apparatus for the residual gas treatment, comprising at least one inlet for hydrogen-containing residual gas, at least one outlet for hydrogen gas, and at least one outlet for end gas, where the inlet for hydrogen-containing residual gas is in fluid connection with the outlet for hydrogen-containing residual gas of the separation unit for separating off carbon monoxide.

A very particularly preferred embodiment of the apparatus is able to recycle CO₂ removed in the apparatus for removal of carbon dioxide to a reactor for the partial oxidation. Therefore, a most preferred embodiment of the apparatus is characterized in that at least one reactor of the apparatus for the partial oxidation has an inlet for carbon dioxide that is in fluid connection with the outlet for carbon dioxide of the apparatus for removal of carbon dioxide, which allows recycling of the carbon dioxide to this reactor of the apparatus for the partial oxidation against the direction of flow of the carbon monoxide-containing product gas stream.

The process regime with an at least two-stage partial oxidation has been found to be preferable. Suitable for this purpose is an embodiment of the apparatus in which said apparatus for partial oxidation comprises
at least one outlet for a carbon monoxide-containing product gas stream
and
a first reactor for the partial oxidation of the material having at least one heating element capable of temperature control of the reactor at a temperature of at least 400° C., where the first reactor comprises at least one inlet for an oxygen-containing gas stream and at least one inlet for material and at least one outlet for product gas that is different therefrom, where the inlet for an oxygen-containing gas stream is in fluid connection with a source of an oxygen-containing gas stream containing at least 50% by weight of oxygen gas;
and
comprises a second reactor for the partial oxidation of the product gas having at least one heating element suitable for temperature control of the reactor at a temperature of more than 800° C., where the second reactor comprises at least one inlet for an oxygen-containing gas stream and at least one inlet for optionally intermediately treated product gas of the first reactor and at least one outlet for product gas that is different therefrom, where the outlet for product gas of the first reactor is in fluid connection with the inlet of the second reactor for optionally intermediately treated product gas of the first reactor, and the inlet for an oxygen-containing gas stream is in fluid connection with a source of an oxygen-containing gas stream containing at least 50% by weight of oxygen gas, and the outlet for product gas of the second reactor is in fluid connection with the outlet for the carbon monoxide-containing product gas stream.

Preferably, a reactor is designed in such a way that, in addition to the oxygen-containing gas stream, additional gaseous water can be introduced into the reactor. Such reactors are characterized in that at least one reactor is in fluid connection with at least one source of gaseous water.

In addition, an embodiment of the apparatus is preferred in which the outlet for carbon monoxide of the separation unit for carbon monoxide is in fluid connection with the inlet of a reactor of an apparatus for phosgene synthesis. The apparatus for phosgene synthesis comprises at least one inlet for chlorine gas, at least one inlet for carbon monoxide, and at least one outlet for phosgene.

For the synthesis of organic isocyanate, it is in a preferred embodiment of the apparatus advantageous when the outlet for phosgene of the apparatus for phosgene synthesis is in fluid connection with an apparatus for producing organic isocyanate, where the apparatus for producing organic isocyanate, in addition to at least one inlet for phosgene, additionally has at least one inlet for organic amine and at least one outlet for organic isocyanate. In this case, it is particularly preferable when the apparatus for producing organic isocyanate is in fluid connection via at least one inlet for organic amine with an apparatus for the hydrogenation of organic nitro compound, where the apparatus for the hydrogenation of organic nitro compound has at least one inlet for hydrogen gas, at least one inlet for organic nitro compound, and at least one outlet for organic amine. An advantageous source of the hydrogen gas for the hydrogenation is the hydrogen gas from the residual gas treatment of the residual gas from the apparatus for the H₂—CO separation. For this purpose, it is preferable that the outlet for hydrogen gas of the apparatus for the residual gas treatment and the inlet for hydrogen gas of the apparatus for the hydrogenation of organic nitro compound are in fluid connection. In addition or as a supplement thereto, a particularly preferable source of the hydrogen gas for the hydrogenation is the hydrogen gas from the apparatus for producing chlorine. For this purpose, it is particularly preferable that the outlet for hydrogen gas of the apparatus for producing chlorine and the inlet for hydrogen gas of the apparatus for the hydrogenation of organic nitro compound are in fluid connection.

For utilization of the hydrogen gas from the residual gas of the apparatus for H₂—CO separation for synthesis purposes it is preferable when the outlet for the residual gas of the apparatus for H₂—CO separation is in fluid connection with the inlet of an apparatus for residual gas treatment, wherein said apparatus for residual gas treatment has at least one outlet for hydrogen gas and at least one outlet for residual gas of the residual gas treatment. In this case it is preferable when the outlet for the residual gas of the apparatus for the residual gas treatment is in fluid connection with the heating element of the reformer, in particular with the combustion chamber of the heating element.

The invention further provides for the use of a partial oxidation of material comprising at least one polymeric organic compound for providing carbon monoxide for the synthesis of phosgene. It is in this case preferable when the partial oxidation of the material is carried out according to a process of the first subject matter of the invention.

The invention further provides for the use of material comprising at least one polymeric organic compound in a partial oxidation of the material for providing carbon monoxide for the synthesis of phosgene. It is in this case preferable when the partial oxidation of the material is carried out according to a process of the first subject matter of the invention.

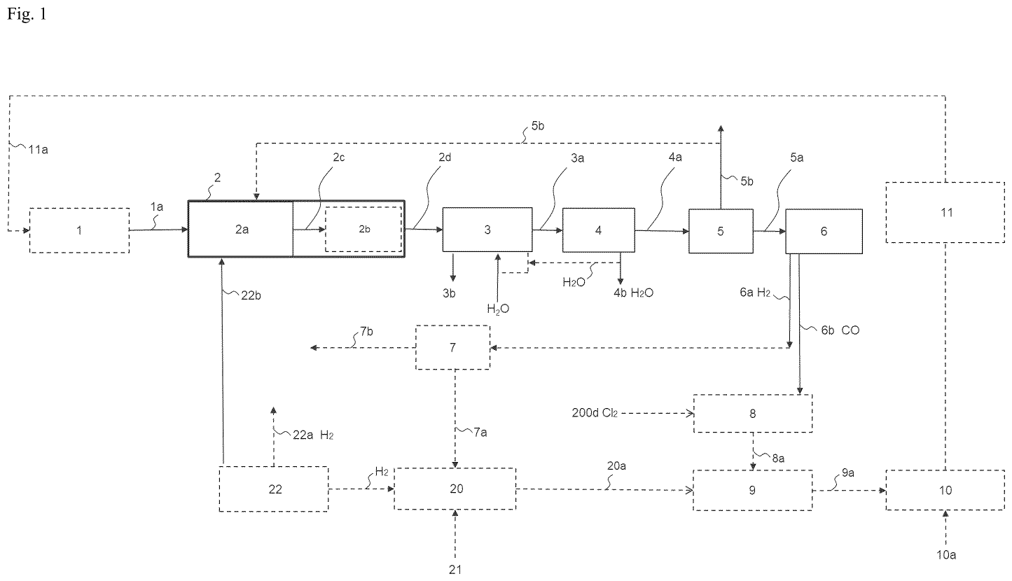

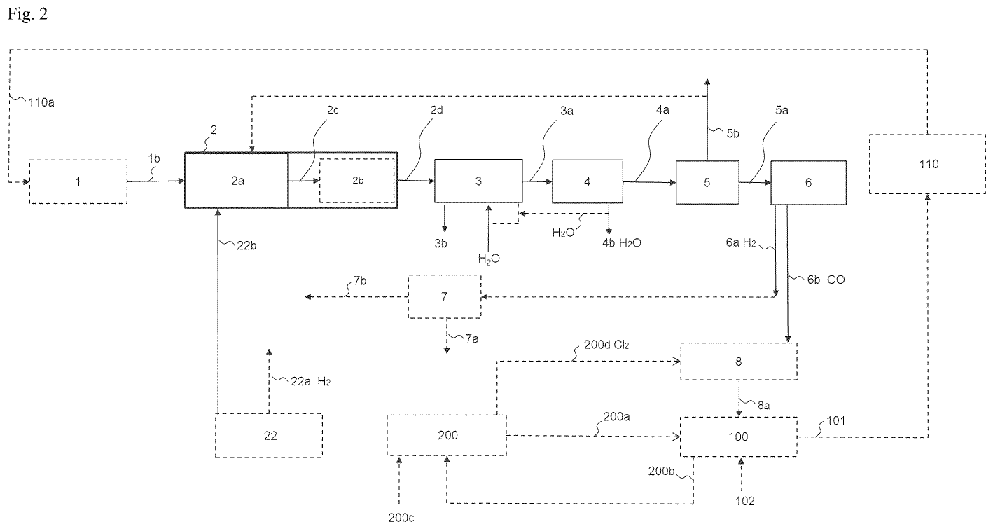

The invention is more particularly elucidated hereinbelow with reference to FIGS. 1 and 2 by way of example. In the figures, the following reference numerals are defined on the right-hand side as follows:
1 Material pretreatment
1a Processed material comprising polyurethane as a polymeric organic compound
1b Processed material comprising polycarbonate as a polymeric organic compound
2 Partial oxidation (gasification)
2a Reactor for partial oxidation
2b Reactor for partial oxidation of the product gas from 2a
2c Product gas
2d Product gas stream containing CO and H₂ and particulate solid
3 Scrubbing step
3a Scrubbed product gas stream containing CO and H₂
3b Slag
4 Drying by removal of H₂O
4a Dried product gas stream containing CO and H₂
4b Water
5 Removal of carbon dioxide
5a Product gas stream containing CO and H₂ after removal of CO₂
5b CO₂
6 Separating off carbon monoxide (separation of H₂ and CO)
6a Residual gas
6b CO
7 H₂ purification
7a H₂
7b End gas
8 Phosgene synthesis
8a Phosgene
9 Isocyanate production
9a Organic isocyanate
10 Polyurethane production
10a Organic compound having at least 2 hydroxy groups, e.g. polyether polyol or polyester polyol
11 End-of-life polyurethane-collection/separation
11a Polyurethane-containing material
20 Hydrogenation of organic nitro compounds
20a Organic diamino compound
20b Organic compound
21 Organic nitro compound
22 Water electrolysis
22a Hydrogen
22b Oxygen
30 H₂O for partial oxidation (gasification) 2
100 Polycarbonate production
101 Polycarbonate
110 End-of-life polycarbonate-containing material-collection/separation
110a Polycarbonate-containing material
200 Chloralkali electrolysis
200a Sodium hydroxide solution
200b Sodium chloride-containing wastewater from 100
200c Sodium chloride (NaCl)
200d Chlorine gas

Arrows in the figures symbolize the flow of substances, energy or heat between process steps/apparatus parts in which the corresponding process steps proceed. Dashed arrows symbolize additional features of preferred embodiments of the process, which can be used with preference individually or in combination.

FIG. 1 shows an embodiment of the process for producing carbon monoxide by gasification of a polyurethane waste fraction using oxygen from a water electrolysis for the preparation of phosgene for the synthesis of organic isocyanate, comprising at least the steps of:
a) providing an oxygen-containing gas stream 22b containing at least 50% by weight of oxygen gas;
b) partially oxidizing 2 material 1a comprising at least one polymeric organic compound, said material 1a being introduced into a reactor 2a of an apparatus and therein being treated at least through the supply of said oxygen-containing gas stream 22b and by heating to at least 400° C. to form a product gas 2c, the resulting product gas 2c, optionally after at least one further partial oxidation step 2b of the product gas 2c, being discharged from the apparatus as a carbon monoxide-containing product gas stream 2d together with particulate solid dispersed therein;
c) supplying the carbon monoxide-containing product gas stream 2d to a purification in which at least
   c1) the carbon monoxide-containing product gas stream 2d is, for the removal of solids, supplied to a scrubbing step 3 in which
      (i) water is brought into contact with the carbon monoxide-containing product gas stream 2d, as a result of which the particulate solid dispersed in the carbon monoxide-containing product gas stream 2d forms a slag 3b and this slag 3b is removed and discharged,
      (ii) the carbon monoxide-containing product gas stream 3a purified of particulate solid is discharged;
   c2) a carbon monoxide-containing product gas stream 3a purified of particulate solid through a scrubbing step 3 is in a drying step at least supplied to a removal of water 4, water 4b is removed, and the resulting carbon monoxide-containing product gas stream 4a is discharged;
   c3) a carbon monoxide-containing product gas stream 4a purified through removal of water 4 is at least supplied to a removal of carbon dioxide 5, carbon dioxide is removed, and the resulting carbon monoxide-containing product gas stream 5a and carbon dioxide 5b is discharged, the carbon dioxide 5b being introduced at least in part into a reactor for the partial oxidation 2;
   c4) a carbon monoxide-containing product gas stream 5a purified through removal of carbon dioxide is at least supplied to a separation unit 6 for separating off carbon monoxide, carbon monoxide is separated off, and the resulting carbon monoxide 6b and a hydrogen gas-containing residual gas 6a is discharged;
   c5) optionally, a hydrogen gas-containing residual gas 6a separated off by means of said separation unit 6 is supplied to a residual gas treatment 7, hydrogen gas 7a is separated off, and hydrogen gas 7a and an end gas 7b is discharged;
d) synthesizing phosgene 8 through reaction of at least chlorine gas and carbon monoxide, where the carbon monoxide that is reacted is at least the carbon monoxide 6b from the purification in step c) and phosgene 8a is discharged.

The integration of this process into a production of isocyanate is described with reference to examples in the examples section.

FIG. 2 shows an embodiment of the process for producing carbon monoxide by gasification of a polycarbonate waste fraction using oxygen from a water electrolysis for the preparation of phosgene for the synthesis of polycarbonate. Which comprises at least the steps described under FIG. 1, except for step d).

The integration of this process into a production of polycarbonate is described with reference to examples in the examples section.

The aspects 1 to 25 that follow represent a further embodiment of the invention, where the reference numerals in brackets are for clarification purposes only, the embodiment not being limited to the subject matter of FIG. 1:

1 A process for providing carbon monoxide for the production of phosgene, preferably for the production of phosgene for the synthesis of organic isocyanate compounds, comprising at least the following steps:
a) providing an oxygen-containing gas stream (22b) containing at least 50% by weight of oxygen gas,
b) partially oxidizing (2) a material (1a) comprising at least one polymeric organic compound, said material (1a) being introduced into a reactor (2a) of an apparatus and therein being treated at least through the supply of said oxygen-containing gas stream (22b) and by heating to at least 400° C. to form a product gas (2c), and the resulting product gas (2c), optionally after at least one further partial oxidation step (2b) of the product gas (2c), being discharged from the apparatus as a carbon monoxide-containing product gas stream (2d) together with particulate solid dispersed therein;
c) supplying the carbon monoxide-containing product gas stream (2d) to a purification in which at least
   c1) the carbon monoxide-containing product gas stream (2d) is, for the removal of solid, supplied to a scrubbing step (3) in which
      (i) water is brought into contact with the carbon monoxide-containing product gas stream (2d), as a result of which the particulate solid dispersed in the carbon monoxide-containing product gas stream (2d) forms a slag (3b) and this slag (3b) is removed and discharged,
      (ii) the carbon monoxide-containing product gas stream (3a) purified of particulate solid is discharged;
   c2) a carbon monoxide-containing product gas stream (3a) purified of particulate solid through a scrubbing step (3) is in a drying step at least supplied to a removal of water (4), water (4b) is removed, and the resulting carbon monoxide-containing product gas stream (4a) is discharged,
   c3) a carbon monoxide-containing product gas stream (4a) purified through removal of water (4) is at least supplied to a removal of carbon dioxide (5), carbon dioxide is removed, and the resulting carbon monoxide-containing product gas stream (5a) and carbon dioxide (5b) is discharged;
   c4) a carbon monoxide-containing product gas stream (5a) purified through removal of carbon dioxide is at least supplied to a separation unit (6) for separating off carbon monoxide, carbon monoxide is separated off, and the resulting carbon monoxide (6b) and a hydrogen gas-containing residual gas (6a) is discharged;
   c5) optionally, a hydrogen gas-containing residual gas (6a) separated off by means of said separation unit (6) is supplied to a residual gas treatment (7), hydrogen gas (7a) is separated off, and hydrogen gas (7a) and an end gas (7b) is discharged;
d) optionally synthesizing phosgene (8) through reaction of at least chlorine gas and carbon monoxide, where the carbon monoxide that is reacted is at least the carbon monoxide (6b) from the purification in step c) and phosgene (8a) is discharged.

2. The process according to aspect 1, characterized in that the oxygen-containing gas stream (22b) is provided by carrying out an electrolysis of water (22) to obtain oxygen gas (22a) and hydrogen gas and the oxygen gas (22a) from this electrolysis (22) is used to provide the oxygen-containing gas stream (22b).

3. The process according to either of aspects 1 or 2, characterized in that the reactor (2a) is additionally supplied with gaseous water in a weight ratio of gaseous water to oxygen gas in the oxygen-containing gas stream of at least 0.2.

4. The process according to any of the preceding aspects, characterized in that the partial oxidation (2) is carried out at an absolute pressure of more than 1 bar, preferably at an absolute pressure within a range from 2 to 80 bar, more preferably at an absolute pressure within a range from 2 to 50 bar.

3. The process according to any of the preceding aspects, characterized in that the total amount of all the polymeric organic compounds present in the material (1a) is at least 50% by weight, preferably of at least 60% by weight, more preferably of at least 75% by weight.

4. The process according to any of the preceding aspects, characterized in that the total amount of all the polymeric organic compounds present in the material (1a) has a carbon content of at least 40.0% by weight, preferably of at least 50% by weight.

5. The process according to any of the preceding aspects, characterized in that the weight ratio of the oxygen gas present in the oxygen-containing gas stream (22b) to the polymeric organic compound are introduced into the reactor (2a) within a weight ratio range of from 0.4:1.0 to 1.2:1.0, preferably from 0.6:1.0 to 0.9:1.0.

6. The process according to any of the preceding aspects, characterized in that the polymeric organic compound in the material (1a) is selected from at least one homopolymer or copolymer compound from the group cellulose, polyester, polyamide, polyurethane (PUR), polyurea, polyisocyanurate (PIR), polycarbonate, preferably from at least one homopolymer or copolymer compound from the group polyurethane (PUR) and polyisocyanurate (PIR).

7. The process according to any of the preceding aspects, characterized in that the structure of the polymeric organic compound in the material (1a) contains at least one repeating structural unit of the formula (I),

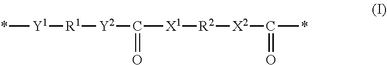

where
X¹ and X² are each independently a direct bond, O, or NH, preferably O or NH,
Y¹ and Y² are each independently O or NH,
R¹ and R² are each independently any divalent carbon-containing structural element, and a covalent bond marked with * denotes a bond to the remainder of the molecule of the polymeric organic compound.

8. The process according to aspect 7, characterized in that the total amount of polymeric organic compounds having at least one repeating structural unit of the formula (I) present in the material (1a) is at least 50% by weight, preferably of at least 60% by weight, more preferably of at least 75% by weight.

9. The process according to any of the preceding aspects, characterized in that the partial oxidation of the material (1a) in the reactor (2a) is carried out at a temperature in a temperature range of from 600° C. to 1500° C., especially from 850° C. to 1400° C., more preferably from 1100° C. to 1300° C.

10. The process according to any of the preceding aspects, characterized in that the water is sprayed into the carbon monoxide-containing product gas stream (2c) during the scrubbing step (3).

11. The process according to any of the preceding aspects, characterized in that the carbon monoxide-containing product gas stream (3a) that has been discharged from the scrubbing step (3) and purified of particulate solid has a temperature of not more than 100° C., preferably of not more than 80° C.

12. The process according to any of the preceding aspects, characterized in that the partial oxidation (2) of the material (1a) is carried out in at least two steps, wherein in a first step a partial oxidation of the material (1a) is carried out in a first reactor (2a) supplied with an oxygen-containing gas stream (22b), at a temperature in a temperature range of 400° C. to 800° C., and the product gas (2c) from the first reactor is transferred to a second reactor (2b) where, in a further step with supply of an oxygen-containing gas stream (22b), it is subjected to a partial oxidation at a temperature of more than 800° C., preferably at a temperature within a range of from 1300° C. to 1500° C., to obtain the carbon monoxide-containing product gas stream (2d).

13. The process according to any of aspects 1 to 12, characterized in that the removed carbon dioxide (5b) discharged in step c3) is recycled to a reactor for the partial oxidation (2) and introduced into this reactor.

14. The process according to aspect 13, characterized in that the partial oxidation (2) of the material (1a) is carried out according to claim 12 and that the removed carbon dioxide (5b) discharged in step c3) is recycled to at least the first reactor (2a) for the partial oxidation and introduced into this reactor.

15. The process according to any of the preceding aspects, characterized in that the carbon dioxide (5b) is added to the partial oxidation (2) in an amount of not more than 1 kg per kg of polymeric organic compound, preferably of not more than 0.9 kg per kg of polymeric organic compound, more preferably of 0.8 kg per kg of polymeric organic compound.

16. The process according to any of the preceding aspects, characterized in that the phosgene synthesis (8) in step d) and additionally the following step is carried out:
e) producing organic isocyanate (9), wherein the phosgene (8a) from the phosgene synthesis (8) is reacted with at least one organic amino compound (20a) and at least organic isocyanate (9a) is discharged.

17. An apparatus for providing carbon monoxide for the production of phosgene, preferably for the production of phosgene for synthesizing organic isocyanate compounds, comprising at least one metering apparatus for feeding in a material, at least one apparatus for the partial oxidation, and at least one purification apparatus for carbon monoxide-containing product gas, characterized in that
i) said apparatus for the partial oxidation comprises
at least one outlet for a carbon monoxide-containing product gas stream
and
at least one reactor for the partial oxidation of the material having at least one heating element suitable for temperature control of the reactor at a temperature of at least 400° C., where the reactor comprises at least one inlet for an oxygen-containing gas stream and at least one inlet for material and at least one outlet for product gas that is different therefrom, where the inlet for an oxygen-containing gas stream is in fluid connection with a source of an oxygen-containing gas stream containing at least 50% by weight of oxygen gas, and the outlet for product gas of the reactor is in fluid connection, optionally at least via at least one interposed reactor for the partial oxidation of the product gas, with the outlet for the carbon monoxide-containing product gas stream; and,
ii) the metering apparatus and said apparatus for the partial oxidation are arranged with respect to one another and configured such that the metering apparatus is in fluid connection, via at least one supply line, with the inlet for material of the reactor for the partial oxidation of the material; and
iii) a purification apparatus for carbon monoxide-containing product gas that comprises at least one outlet for carbon monoxide and at least one inlet for the carbon monoxide-containing product gas stream, said inlet being in fluid connection with the outlet for the carbon monoxide-containing product gas stream of the apparatus for the partial oxidation, the purification apparatus being configured such that the carbon monoxide-containing product gas stream can be passed for purification through modules listed below in the specified order of priority;
   iii-1) at least one gas-scrubbing apparatus, comprising at least one inlet for the carbon monoxide-containing product gas stream, at least one inlet for water, at least one scrubbing tank in which water and carbon monoxide-containing product gas stream are brought together, and at least one outlet for carbon monoxide-containing product gas stream and at least one outlet for slag, where the scrubbing tank and the outlet for slag are in fluid connection and the scrubbing tank and the outlet for carbon monoxide-containing product gas stream are in fluid connection;
   iii-2) at least one drying apparatus, comprising at least one inlet for carbon monoxide-containing product gas stream, at least one cooling apparatus suitable for cooling the carbon monoxide-containing product gas stream, at least one outlet for carbon monoxide-containing product gas stream, and at least one outlet for liquid condensate,
   iii-3) at least one apparatus for removal of carbon dioxide, comprising at least one inlet for carbon monoxide-containing product gas stream, at least one outlet for carbon monoxide-containing product gas stream and, in addition, at least one outlet for carbon dioxide,
   iii-4) at least one separating unit for separating off carbon monoxide, comprising at least one inlet for carbon monoxide-containing product gas stream, at least one outlet for carbon monoxide and, in addition, at least one outlet for hydrogen gas-containing residual gas.

18. The apparatus according to aspect 17, characterized in that the purification apparatus additionally comprises at least one apparatus for the residual gas treatment, comprising at least one inlet for hydrogen-containing residual gas, at least one outlet for hydrogen gas, and at least one outlet for end gas, where the inlet for hydrogen-containing residual gas is in fluid connection with the outlet for hydrogen-containing residual gas of the separation unit for separating off carbon monoxide.

19. The apparatus according to either of aspects 17 or 18, characterized in that at least one reactor of the apparatus for the partial oxidation has an inlet for carbon dioxide that is in fluid connection with the outlet for carbon dioxide of the apparatus for removal of carbon dioxide, which allows recycling of the carbon dioxide to this reactor of the apparatus for the partial oxidation against the direction of flow of the carbon monoxide-containing product gas stream.

20. The apparatus according to any of aspects 17 to 19, characterized in that said apparatus for partial oxidation comprises at least one outlet for a carbon monoxide-containing product gas stream and comprises a first reactor for the partial oxidation of the material having at least one control suitable for the setting of temperature of the reactor to a temperature of at least 400° C., where the first reactor comprises at least one inlet for an oxygen-containing gas stream and at least one inlet for material and at least one outlet for product gas that is different therefrom, where the inlet for an oxygen-containing gas stream is in fluid connection with a source of an oxygen-containing gas stream containing at least 50% by weight of oxygen gas;
and
comprises a second reactor for the partial oxidation of the product gas having at least one heating element suitable for temperature control of the reactor at a temperature of more than 800° C., where the second reactor comprises at least one inlet for an oxygen-containing gas stream and at least one inlet for optionally intermediately treated product gas of the first reactor and at least one outlet for product gas that is different therefrom, where the outlet for product gas of the first reactor is in fluid connection with the inlet of the second reactor for optionally intermediately treated product gas of the first reactor, and the inlet for an oxygen-containing gas stream is in fluid connection with a source of an oxygen-containing gas stream containing at least 50% by weight of oxygen gas, and the outlet for product gas of the second reactor is in fluid connection with the outlet for the carbon monoxide-containing product gas stream.

21. The apparatus according to any of aspects 17 to 20, characterized in that at least one reactor is in fluid connection with at least one source of gaseous water.

22. The apparatus according to any of aspects 17 to 21, characterized in that the outlet for carbon monoxide of the separation unit for carbon monoxide is in fluid connection with the inlet of a reactor of an apparatus for phosgene synthesis.

23. The use of a partial oxidation of material (2) comprising at least one polymeric organic compound for providing carbon monoxide for the synthesis of phosgene.

24 The use of material (2) comprising at least one polymeric organic compound in a partial oxidation of the material for providing carbon monoxide for the synthesis of phosgene.

25. The use according to either of aspects 23 or 24, characterized in that the partial oxidation of the material is carried out by a process according to any of aspects 1 to 16.

### Examples

### Example 1-Inventive process-partial oxidation 2 of polyurethane-containing material (two reaction zones 2a and 2b)—without recycling of CO₂ according to FIG. 1

Into a reactor 2a of an apparatus for a partial oxidation 2 is introduced 10.94 t/h of polyurethane-containing material 1a having a composition of 54.2% by weight of carbon, 8.78% by weight of hydrogen, 35.7% by weight of oxygen, and 1.1% by weight of N₂ and also 0.5% by weight of non-combustible inorganic solids. The polyurethane-containing material 1a undergoes reaction in reactor 2a at 950° C., resulting in a CO— and H₂-containing product gas 2c that is supplied to a reactor 2b. In reactor 2b the gas mixture 2c introduced from 2a undergoes reaction at 1450° C.

In addition, 5.46 t/h of oxygen 22b from a water electrolysis 22 is supplied to the partial oxidation 2.

The CO— and H₂-containing gas mixture 2d from zone 2 2b is in a scrubbing step 3 contacted with water and cooled to 60° C. This is accompanied by the removal of 0.049 t/h of slag 3b, 0.1 t/h of water, and 0.54 t/h of ash. A part-amount of the condensed water is purged and replaced by fresh water.

The scrubbed product gas mixture 3a from the partial oxidation 2 is supplied to a drying 4.

The dried product gas mixture 4a is supplied to an amine scrubbing for removal of CO₂ 5, in which 7.9 t/h of CO₂ 5b is removed. The product gas mixture 5a largely freed of CO₂ is supplied to a coldbox for H₂—CO separation 6, in which the remaining CO₂ is also removed. 6.28 t/h of CO 6b and 0.95 t/h of H₂-containing residual gas 6a and small amounts of by-products, including methane and nitrogen, are withdrawn from the coldbox.

The CO 6b is supplied to a phosgene synthesis 8, where it is reacted with 15.92 t/h of Cl₂ to form 22.2 t/h of phosgene 8a. The phosgene 8a is reacted with 13.68 t/h of toluenediamine in an isocyanate synthesis 9 to form 19.5 t/h of toluene diisocyanate 9a. This can be processed further into polyurethane materials using polyols/polyesterols 10a.

After utilization of the polyurethane materials in various uses, these end-of-life materials 110 are collected, separated, and processed, affording a material 1b that can be supplied to the gasification 2. Thus, the CO group of the isocyanate functional group (—NCO) in the isocyanate can be sustainably produced and the carbon recycled, thereby closing the value chain for this carbon.

The hydrogen-containing residual gas stream 6a separated from the coldbox 6 is supplied to a pressure-swing adsorption unit 7 for purification of the hydrogen and 0.9 t/h of H₂ separated therefrom and supplied to the hydrogenation of dinitrotoluene 20. In the hydrogenation, 20.4 t/h of dinitrotoluene 21 is reacted with the 0.9 t/h of H₂ 7a from the residual gas treatment 7 plus a further 0.445 t/h of H₂ from a water electrolysis 22 to form 13.68 t/h of toluenediamine 20a.

With the co-product hydrogen that is used for the hydrogenation of nitro compounds, a large part of the required hydrogen can be provided, which then no longer has to be produced by water electrolysis and thus likewise provides a sustainable contribution.

The 3.6 t/h of oxygen 22b withdrawn from the water electrolysis 22 as a co-product is here supplied to the gasification 2 with an additional 1.86 t/h of oxygen.

### Example 2 FIG. 1-Inventive process-partial oxidation of polyurethane-containing material (two reaction zones 2a and 2b) with recycling of CO₂

Into an apparatus for a partial oxidation 2 is introduced 10.94 t/h of polyurethane-containing material 1a having an average composition of 54.2% by weight of carbon, 8.73% by weight of hydrogen, 35.5% by weight of oxygen, 1.1% by weight of N₂, and also 0.45% by weight of non-combustible inorganic solids. The polyurethane-containing material 1a undergoes reaction in the apparatus in a zone 2a with the addition of oxygen-containing gas 22b at 950° C., from a CO—and H₂-containing gas mixture is supplied to a zone 2 2b. In zone 2 2b the product gas mixture 2c introduced from 2a undergoes reaction at 1450° C. In addition, 5.46 t/h of oxygen 22b from a water electrolysis 22 and 7.91 t/h of CO₂ 5b from the CO₂ removal unit 5 are supplied to the gasification apparatus (2). The CO—and H₂-containing gas mixture 2d is in a scrubbing step 3 cooled to 60° C. by scrubbing with water. This is accompanied by the removal of 0.049 t/h of slag 3b, 0.1 t/h of water, and 0.54 t/h of ash. A 3.23 t/h part-amount of the condensed water is purged and 0.1 t/h of fresh water added.

After cooling, the product gas mixture 3a from the scrubbing step 3 is supplied to a drying 4.

The dried gas mixture 4a is supplied to an amine scrubbing for removal of CO₂ 5, in which 7.91 t/h of CO₂ 5b is removed. The gas mixture largely freed of CO₂ is supplied to a coldbox for H₂—CO separation 6, in which the remaining CO₂ is also removed. 11.31 t/h of CO 6b and 0.6 t/h of H₂-containing residual gas 6a and small amounts of by-products, including methane and nitrogen, are withdrawn from the coldbox. The CO 6b is supplied to a phosgene synthesis 8, where it is reacted with 28.67 t/h of Cl₂ to form 39.98 t/h of phosgene 8a. The phosgene 8a is reacted with 24.63 t/h of toluenediamine 20a in an isocyanate synthesis 9 to form 35.13 t/h of toluene diisocyanate 9a. This can be processed further into polyurethane materials using polyols/polyesterols 10a. After utilization of the polyurethane materials in various uses, these are collected, separated, and processed 1 as end-of-life material 11a, affording a material 1a that can be supplied to the partial oxidation 2. Thus, the CO group of the isocyanate functional group (—NCO) in the isocyanate can be sustainably produced and the carbon recycled, thereby closing the value chain for this carbon.

The hydrogen-containing residual gas stream 6a separated from the coldbox 6 is supplied to a pressure-swing adsorption unit 7 for purification of the residual gas and 0.55 t/h of H₂ 7a separated therefrom supplied to the to the hydrogenation of dinitrotoluene 20. In the hydrogenation, 34.74 t/h of dinitrotoluene 21 is reacted with the 0.55 t/h of H₂ 7a from the hydrogen purification unit plus a further 1.87 t/h of H₂ from a water electrolysis 22 to form 24.63 t/h of toluenediamine 20a.

With the co-product hydrogen that is used for the hydrogenation of nitro compounds, a large part of the required hydrogen can be provided, which then no longer has to be produced by water electrolysis and thus likewise represents a sustainable contribution.

Of the total of 14.97 t/h of oxygen 22b formed as a co-product in the water electrolysis 22, 5.46 t/h is supplied to the partial oxidation 2.

### Example 3, FIG. 2-Inventive process-Gasification in a polycarbonate composite-without recycling of CO₂

Into a gasification apparatus 2 is introduced 10.935 t/h of polycarbonate-containing material 1b having an average composition of 75.65% by weight of carbon, 5.51% by weight of hydrogen, 18.9% by weight of oxygen, and also 0.45% by weight of non-combustible inorganic solids. The polycarbonate-containing material 1b undergoes reaction in the gasification apparatus 2 in a zone 2a at 950° C., from a CO—and H₂-containing product gas mixture 2c is supplied to a zone 2 2b and 0.54 t/h of ash is discharged (not depicted). In 2b the gas mixture 2c introduced from 2a undergoes reaction at 1450° C. In addition, 9.89 t/h of oxygen 22b from a water electrolysis 22 is supplied to the gasification apparatus. The CO—and H₂-containing product gas mixture 2d from 2b is in a scrubbing step 3 cooled to 60° C. by scrubbing with water. This is accompanied by the removal of 0.049 t/h of slag 3b and 0.1 t/h of water. A 1.6 t/h part-amount of the condensed water is purged and replaced by 1.7 t/h of fresh water.

The cooled product gas mixture 3a from scrubbing step 3 is supplied to a drying 4.

The dried product gas mixture 4a is supplied to an amine scrubbing for removal of CO₂ 5, in which 6.52 t/h of CO₂ 5b is removed. The gas mixture 5a largely freed of CO₂ is supplied to a coldbox for H₂—CO separation 6, in which the remaining CO₂ is also removed. 12.61 t/h of CO 6b and 0.6 t/h of H₂-containing residual gas 6a and small amounts of by-products, including methane, are withdrawn from the coldbox.

The hydrogen-containing residual gas stream 6a separated from the coldbox is supplied to a pressure-swing adsorption unit 7 for purification of the hydrogen and 0.55 t/h of H₂ 7a separated therefrom and supplied to a further recovery.

The CO from the coldbox is supplied to a phosgene synthesis 8, where it is reacted with 31.97 t/h of Cl₂ to form 44.58 t/h of phosgene 8a.

44.58 t/h of phosgene 8a is withdrawn from the phosgene synthesis 8 and in a polycarbonate production 100, as a solution in chlorinated solvent (for example 482 t/h of a 1:1 mixture of methylene chloride and chlorobenzene) (2), reacted with 599.4 t/h of 15% by weight bisphenol A solution 102 (89.9 t/h of BPA, equivalent to 393.8 kmol) in alkaline water (containing 2.13 mol of NaOH per mol of bisphenol A, equivalent to 838.8 kmol of NaOH=33.5 t/h of NaOH and 71.2 t of H₂O as 32% by weight sodium hydroxide solution)) to afford 102.9 t/h of polycarbonate 101 using at least one chain terminator (for example 2.55 t/h of tert-butylphenol), 32.19 t/h of further 22% by weight NaOH solution (7.08 t/h of NaOH and 19.8 t/h of H₂O as 32% sodium hydroxide solution), and at least one catalyst (for example 0.5 t/h of N-ethylpiperidine). 36.0 t/h of NaOH with 76.5 t/h of water are obtained as 32% by weight sodium hydroxide solution 200a from the chloralkali electrolysis 200. A supplementary amount of 4.58 t/h of NaOH with 19.8 t/h of H₂O is additionally supplied.

The polycarbonate production uses 627.2 t/h of scrubbing/process water, which forms inter alia the NaCl-containing wastewater 200b.

The resulting NaCl-containing wastewater comprising an amount of 59.38 t/h of NaCl and 1128.3 t/h of water is obtained as NaCl-containing wastewater. A substream consisting of 0.85 t/h of NaCl and 16.2 t/h of H₂O 200b is, after purification by a stripping plant with entraining gas and an activated carbon purification, supplied to a Cl₂ production by chloralkali electrolysis 200. This covers the water demand of the electrolysis, thus obviating the need for addition of water to the chloralkali electrolysis. This conserves water resources. The polycarbonate 101 obtained is used for the synthesis of polycarbonate material for commercial uses.

After utilization of the polycarbonate material in various commercial uses, said material can be collected as end-of-life material 110a and recycled, in order to supply the polycarbonate material waste obtained therefrom after processing 1 to a gasification apparatus as a material for partial oxidation 1b.

The process of the invention makes it possible to substitute 6.25% of the carbon present in the PC from a non-fossil carbon source. The use of renewable energy in the chloralkali electrolysis further reduces the CO₂ footprint of the phosgene produced from CO and Cl₂, thus allowing for a sustainably produced PC.

### Example 4, FIG. 2-Inventive process-Gasification in a polycarbonate composite—with recycling of CO₂

Into a gasification apparatus 2 is introduced 10.935 t/h of polycarbonate-containing material 1b having an average composition of 75.65% by weight of carbon, 5.51% by weight of hydrogen, 18.9% by weight of oxygen, and also 0.45% by weight of non-combustible inorganic solids. The polyurethane-containing material 1b undergoes reaction in the gasification apparatus 2 in a zone 2a at 950° C., from a CO—and H₂-containing gas mixture 2c is supplied to a zone 2 2b and 0.54 t/h of ash is discharged. In 2b the gas mixture 2c introduced from 2a undergoes reaction at 1450° C. In addition, 9.89 t/h of oxygen from a water electrolysis and 6.52 t/h of CO₂ from the CO₂ removal unit are supplied to the gasification apparatus 2. The CO—and H₂-containing gas mixture from zone 2 is cooled to 60° C. by quenching with water. This is accompanied by the removal of 0.049 t/h of slag and 0.1 t/h of water. A 2.67 t/h part-amount of the condensed water is purged and 0.1 t/h of fresh water added.

After cooling, the gas mixture from the gasification apparatus 2 is supplied to a drying.

The dried gas mixture is supplied to an amine scrubbing for removal of CO₂, in which 6.52 t/h of CO₂ is removed. The CO₂ is returned to the gasification apparatus. The gas mixture largely freed of CO₂ is supplied to a coldbox for H₂—CO separation, in which the remaining CO₂ is also removed. 16.77 t/h of CO and 0.31 t/h of H₂ and small amounts of by-products, including methane, are withdrawn from the coldbox.

The hydrogen-containing gas stream separated from the coldbox 6 is supplied to a pressure-swing adsorption unit 7 for purification of the hydrogen and 0.28 t/h of H₂ separated therefrom and supplied to a further recovery.

The CO 6b from the coldbox 6 is supplied to a phosgene synthesis 8, where it is reacted with 42.52 t/h of Cl₂ to form 59.29 t/h of phosgene 8a.

44.58 t/h of phosgene 8a is withdrawn from the phosgene synthesis 8 and in a polycarbonate production 100, as a solution in chlorinated solvent (for example 641 t/h of a 1:1 mixture of methylene chloride and chlorobenzene) (2), reacted with 797.2 t/h of 15% by weight bisphenol A solution 102 (119.56 t/h of BPA, equivalent to 523.74 kmol) in alkaline water (containing 2.13 mol of NaOH per mol of bisphenol A, equivalent to 1115.6 kmol of NaOH=44.55 t/h of NaOH and 94.69 t/h of H₂O as 32% by weight sodium hydroxide solution)) as diol to afford 136.85 t/h of polycarbonate 101 using at least one chain terminator (for example 3.39 t/h of tert-butylphenol), 42.8 t/h of further 22% by weight NaOH solution (9.4 t/h of NaOH and 26.3 t/h of H₂O as 32% sodium hydroxide solution), and at least one catalyst (for example 0.66 t/h of N-ethylpiperidine). 47.88 t/h of NaOH with 101.75 t/h of water are obtained as 32% by weight sodium hydroxide solution from the chloralkali electrolysis 200. An additional amount of 6.09 t/h of NaOH with 26.33 t/h of H₂O is purchased externally and supplied.

The polycarbonate production uses 834.17 t/h of scrubbing/process water, which forms inter alia the NaCl-containing wastewater.

The resulting NaCl-containing wastewater 200b comprising an amount of 78.97 t/h of NaCl and 1500.6 t/h of water is obtained as NaCl-containing wastewater. A substream consisting of 1.13 t/h of NaCl and 21.55 t/h of H₂O is, after purification by a stripping plant with entraining gas and an activated carbon purification, supplied to a Cl₂ production by chloralkali electrolysis 200. This covers the water demand of the electrolysis, thus obviating the need for addition of water to the chloralkali electrolysis 200. This conserves water resources. The polycarbonate 101 obtained is used for the synthesis of polycarbonate material for commercial uses.

After utilization of the polycarbonate material in various commercial uses 110, said material can be collected and recycled, in order to supply the polycarbonate material waste obtained therefrom after processing 1 to a gasification apparatus as a material for partial oxidation 1b.

The process of the invention makes it possible to substitute 6.25% of the carbon present in the polycarbonate from a non-fossil carbon source. The use of renewable energy in the chloralkali electrolysis further reduces the CO₂ footprint of the phosgene produced from CO and Cl₂, thus allowing for a sustainably produced polycarbonate.

1. A process for providing carbon monoxide for the production of phosgene, comprising at least the following steps:
a) providing an oxygen-containing gas stream containing at least 50% by weight of oxygen gas based on the total amount of the oxygen-containing gas stream,
b) partially oxidizing a material comprising at least one polymeric organic compound, said material being introduced into a reactor of an apparatus and therein being treated at least through the supply of said oxygen-containing gas stream and by heating to at least 400° C. to form a product gas, and the resulting product gas, optionally after at least one further partial oxidation step of the product gas, being discharged from the apparatus as a carbon monoxide-containing product gas stream together with particulate solid dispersed therein;
c) supplying the carbon monoxide-containing product gas stream to a purification in which at least:
c1) the carbon monoxide-containing product gas stream is, for the removal of solid, supplied to a scrubbing step in which:
(i) water is brought into contact with the carbon monoxide-containing product gas stream, as a result of which the particulate solid dispersed in the carbon monoxide-containing product gas stream forms a slag and this slag is removed and discharged, and
(ii) a carbon monoxide-containing product gas stream purified of particulate solid is discharged;
c2) a carbon monoxide-containing product gas stream purified of particulate solid through the scrubbing step is at least supplied to a drying step for a removal of water, water is removed, and a resulting carbon monoxide-containing product gas stream purified through removal of water is discharged,
c3) the carbon monoxide-containing product gas stream purified through removal of water is at least supplied to a removal of carbon dioxide, carbon dioxide is removed, and a resulting carbon monoxide-containing product gas stream purified through removal of carbon dioxide and a removed carbon dioxide stream is are discharged;
c4) the carbon monoxide-containing product gas stream purified through removal of carbon dioxide is at least supplied to a separation unit for separating off carbon monoxide, carbon monoxide is separated off, and a resulting carbon monoxide stream and a hydrogen gas-containing residual gas stream is are discharged;
c5) optionally, a hydrogen gas-containing residual gas separated off by means of said separation unit is supplied to a residual gas treatment, hydrogen gas is separated off, and hydrogen gas and an end gas is discharged;
d) optionally synthesizing phosgene through reaction of at least chlorine gas and carbon monoxide, where the carbon monoxide that is reacted is at least the carbon monoxide from the purification in step c) and phosgene is discharged.

2. The process as claimed in claim 1, wherein the oxygen-containing gas stream is provided by carrying out an electrolysis of water to obtain oxygen gas and hydrogen gas, and that wherein the oxygen gas from this electrolysis is used to provide the oxygen-containing gas stream.

3. The process as claimed in claim 1, wherein the reactor is additionally supplied with gaseous water in a weight ratio of gaseous water to oxygen gas in the oxygen-containing gas stream of at least 0.2.

4. The process as claimed in claim 1, wherein the total amount of all the polymeric organic compounds present in the material is at least 50% by weight based on the total amount of the material.

5. The process as claimed in claim 1, wherein the weight ratio of the oxygen gas present in the oxygen-containing gas stream to the polymeric organic compound introduced into the reactor is within a weight ratio range of from 0.4:1.0 to 1.2:1.0.

6. The process as claimed in claim 1, wherein the polymeric organic compound in the material is selected from at least one homopolymer or copolymer compound from the group cellulose, polyester, polyamide, polyurethane, polyurea, polyisocyanurate, polycarbonate.

7. The process as claimed in claim 1, wherein the structure of the polymeric organic compound in the material contains at least one repeating structural unit of the formula, 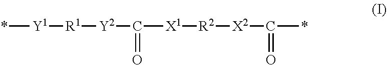
where
X¹ and X² are each independently a direct bond, O, or NH,
Y¹ and Y² are each independently O or NH,
R¹ and R² are each independently any divalent carbon-containing structural element, and
a covalent bond marked with * denotes a bond to the remainder of the molecule of the polymeric organic compound.

8. The process as claimed in claim 7, wherein the total amount of polymeric organic compounds having at least one repeating structural unit of the formula present in the material is at least 50% by weight based on the total amount of the material.

9. The process as claimed in claim 1, wherein the partial oxidation of the material in the reactor is carried out at a temperature in a temperature range of from 600° C. to 1500° C.

10. The process as claimed in claim 1, wherein the carbon monoxide-containing product gas stream that has been discharged from the scrubbing step- and purified of particulate solid has a temperature of not more than 100° C.

11. The process as claimed in claim 1, wherein the partial oxidation of the material is carried out in at least two steps, wherein in a first step a first partial oxidation of the material is carried out in a first reactor supplied with an oxygen-containing gas stream, at a temperature in a temperature range of 400° C. to 800° C., and the product gas from the first reactor is transferred to a second reactor where, in a further step with supply of an oxygen-containing gas stream, the material is subjected to a second partial oxidation at a temperature of more than 800° C., to obtain the carbon monoxide-containing product gas stream.

12. The process as claimed in claim 1, wherein the removed carbon dioxide discharged in step c is recycled to the reactor for the partial oxidation and introduced into the reactor.

13. The process as claimed in claim 11, wherein the removed carbon dioxide discharged in step c is recycled to at least the first reactor for the partial oxidation and introduced into this the first reactor.

14. The process as claimed in claim 1, wherein the removed carbon dioxide is added to the partial oxidation in an amount of not more than 1 kg per kg of the polymeric organic compound.

15. The process as claimed in claim 1, wherein the process comprises the phosgene synthesis in step d) and the process further comprising:
e) producing organic isocyanate, wherein the phosgene from the phosgene synthesis is reacted with at least one organic amino compound and at least organic isocyanate is discharged.

16. An apparatus for providing carbon monoxide for the production of phosgene comprising a metering apparatus for feeding in a material, an apparatus for the partial oxidation, and a purification apparatus for carbon monoxide-containing product gas, wherein:
i) said apparatus for the partial oxidation comprises:
at least one outlet for a carbon monoxide-containing product gas stream; and
at least one reactor for the partial oxidation of the material, the at least one reactor having at least one heating element capable of temperature control of the reactor at a temperature of at least 400° C., at least one inlet for an oxygen-containing gas stream, and at least one inlet for the material and at least one outlet for product gas that is different therefrom, where the inlet for the oxygen-containing gas stream is in fluid connection with a source of an oxygen-containing gas stream containing at least 50% by weight of oxygen gas based on the total amount of the oxygen-containing gas stream, and the outlet for product gas of the at least one reactor is in fluid connection, optionally at least via at least one interposed reactor for the partial oxidation of the product gas, with the outlet for the carbon monoxide-containing product gas stream; and,
ii) the metering apparatus and said apparatus for the partial oxidation are arranged with respect to one another and configured such that the metering apparatus is in fluid connection, via at least one supply line, with the inlet for the material of the at least one reactor for the partial oxidation of the material; and
iii) the purification apparatus for carbon monoxide-containing product gas that comprises at least one outlet for carbon monoxide and at least one inlet for the carbon monoxide-containing product gas stream, said inlet being in fluid connection with the outlet for the carbon monoxide-containing product gas stream of the apparatus for the partial oxidation, the purification apparatus being configured such that the carbon monoxide-containing product gas stream can be passed for purification through modules listed below in the specified order of priority;
iii-1) at least one gas-scrubbing apparatus, comprising at least one inlet for the carbon monoxide-containing product gas stream, at least one inlet for water, at least one scrubbing tank in which water and carbon monoxide-containing product gas stream are brought together, and at least one outlet for carbon monoxide-containing product gas stream and at least one outlet for slag, where the scrubbing tank and the outlet for slag are in fluid connection and the scrubbing tank and the outlet for carbon monoxide-containing product gas stream are in fluid connection;
iii-2) at least one drying apparatus, comprising at least one inlet for carbon monoxide-containing product gas stream, at least one cooling apparatus suitable for cooling the carbon monoxide-containing product gas stream, at least one outlet for carbon monoxide-containing product gas stream, and at least one outlet for liquid condensate,
iii-3) at least one apparatus for removal of carbon dioxide, comprising at least one inlet for carbon monoxide-containing product gas stream, at least one outlet for carbon monoxide-containing product gas stream and, in addition, at least one outlet for carbon dioxide, and
iii-4) at least one separating unit for separating off carbon monoxide,
comprising at least one inlet for carbon monoxide-containing product gas stream, at least one outlet for carbon monoxide and, in addition, at least one outlet for hydrogen gas-containing residual gas.

17. The apparatus as claimed in claim 16, wherein at least one reactor of the apparatus for the partial oxidation has an inlet for carbon dioxide that is in fluid connection with the outlet for carbon dioxide of the apparatus for removal of carbon dioxide, which allows recycling of the carbon dioxide to this reactor of the apparatus for the partial oxidation against a direction of flow of the carbon monoxide-containing product gas stream.

18. The apparatus as claimed in either of claim 16, wherein said apparatus for the partial oxidation comprises:
a first reactor for the partial oxidation of the material having at least one control suitable for the setting of temperature of the first reactor to a temperature of at least 400° C., where the first reactor comprises at least one inlet for an oxygen-containing gas stream and at least one inlet for material and at least one outlet for product gas that is different therefrom, where the inlet for an oxygen-containing gas stream is in fluid connection with a source of an oxygen-containing gas stream containing at least 50% by weight of oxygen gas based on the total amount of the oxygen-containing gas stream; and
a second reactor for the partial oxidation of the product gas having at least one heating element suitable for temperature control of the second reactor at a temperature of more than 800° C., where the second reactor comprises at least one inlet for the oxygen-containing gas stream and at least one inlet for optionally intermediately treated product gas of the first reactor and at least one outlet for product gas that is different therefrom, where the outlet for product gas of the first reactor is in fluid connection with the inlet of the second reactor for optionally intermediately treated product gas of the first reactor, and the at least one inlet for the oxygen-containing gas stream of the second reactor is in fluid connection with the source of the oxygen-containing gas stream containing at least 50% by weight of oxygen gas, and the outlet for product gas of the second reactor is in fluid connection with the outlet for the carbon monoxide-containing product gas stream.

19. The apparatus as claimed in claim 16, wherein the outlet for carbon monoxide of the separation unit for carbon monoxide is in fluid connection with the inlet of a reactor of an apparatus for phosgene synthesis.

20. A method for synthesizing phosgene, comprising providing carbon monoxide using a partial oxidation of a material comprising at least one polymeric organic compound.
